(19) United States
(12) Patent Application Publication
(10) Pub. No.: US 2010/0240611 A1
(43) Pub. Date: Sep. 23, 2010

(54) METHODS FOR PREPARING DPP-IV INHIBITOR COMPOUNDS

(76) Inventors: Matthew Ronsheim, Port Jefferson, NY (US); Nhut Diep, Hauppage, NY (US); Yuriy Kalyan, Staten Island, NY (US); Graham Lawton, Holbrook, NY (US); Peng Wang, Carlsbad, CA (US); Michael Ouellette, San Diego, CA (US)

Correspondence Address: Forest Laboratories, Inc.;Attn: Charles S. Ryan, 500 COMMACK ROAD, Commack, NY, 11725, US

(21) Appl. No.: 12/724,775

(22) Filed: Mar. 16, 2010

Related U.S. Application Data
(60) Provisional application No. 61/160,916, filed on Mar. 17, 2009.
(60) Provisional application No. 61/232,604, filed on Aug. 10, 2009.

(51) Int. Cl. A61K 31/69; C07D 403/12; A61P 3/10

(52) U.S. Cl. 514/64; 548/405

(57) ABSTRACT

Methods for preparing an inhibitor of dipeptidyl peptidase IV, as well as formulations of such inhibitors of dipeptidyl peptidase IV that have a high degree of stability including under warm, humid storage conditions.

### RELATED APPLICATIONS

This application claims the benefit of U.S. Provisional Application No. 61/160,916, filed Mar. 17, 2009, and U.S. Provisional Application No. 61/232,604, filed Aug. 10, 2009. The entire contents of the '916 and '604 Applications are hereby incorporated by reference.

### FIELD OF THE INVENTION

The present invention provides methods for preparing an inhibitor of dipeptidyl peptidase IV, as well as stable formulations of such inhibitors of dipeptidyl peptidase IV, and methods of using such inhibitors.

### BACKGROUND OF THE INVENTION

The enzyme dipeptidyl peptidase IV (DPP-IV) is a member of the dipeptidyl peptidase family, which cleaves N-terminal dipeptide residues from proteins, particularly where the dipeptide includes an N-terminal penultimate proline or alanine residue. DPP-IV is believed to be involved in glucose control, as its peptidolytic action inactivates the insulotropic peptides glucagon-like peptide I (GLP-I) and gastric inhibitory protein (GIP).

Inhibition of DPP-IV, such as with synthetic inhibitors in vivo, can serve to increase plasma concentrations of GLP-I and GIP, and thus improve glycemic control in the body. Such synthetic inhibitors would therefore be useful in the treatment of diabetes mellitus and related conditions. Certain such selective DPP-IV inhibitors have been developed, as are disclosed in U.S. Pat. No. 7,317,109, U.S. Pat. No. 7,576,121, U.S. Application Publication Nos. 2007/0060547, 2007/0185061, 2007/0299036, 2008/0182995, 2008/0300413, 2006/0264400, and 2006/0264401, and in International Applications WO2008/027273 and WO2008/144730, the contents of which are incorporated herein by reference. Inhibition of DPP-IV by compounds of the structure of formula (I) is disclosed therein:

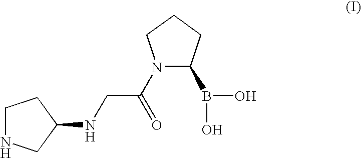

While some methods for preparing the DPP-IV inhibitor of formula (I) have been disclosed in the art, there remains a need for additional methods for preparing such compounds. The present invention seeks to provide such methods.

### SUMMARY OF THE INVENTION

In one embodiment, the present invention relates to a method for preparing the compound of formula (I):

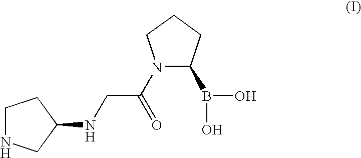

or a pharmaceutically acceptable salt thereof, wherein the method comprises:

(a) coupling a boronic ester of formula (IX) with an acid of formula (V), to form the compound of formula (X):

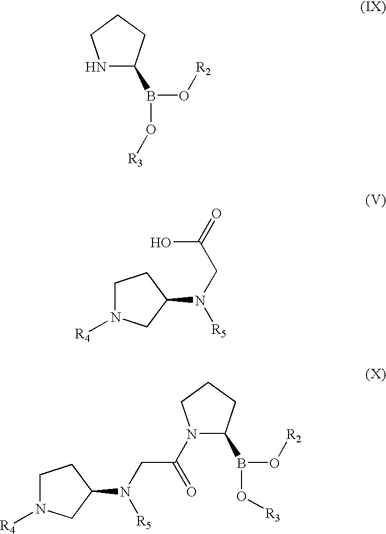

wherein R2, R3, R4 and R5 are protecting groups,

(b) removing the R4 and R5 groups from the compound of formula (X) to form the compound of formula (XI);

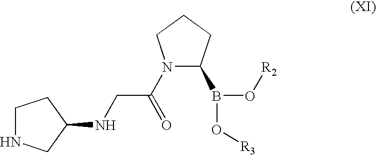

(c) reacting the compound of (XI) with an acid (e.g., a boronic acid) to form the compound of formula (I) and optionally the compound of formula (VII);

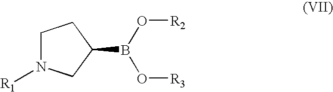

wherein R1 is a protecting group;

(d) optionally, if any compound of formula (VII) is formed in reacting step (c), removing the R1 group from the compound of formula (VII) to form the compound of formula (IX); and

(e) optionally recycling the compound of formula (IX) for use in reacting step (a).

In other embodiments, the present invention relates to a method for preparing the compound of formula (I), or a pharmaceutically acceptable salt thereof, wherein the method comprises:

(a) coupling a boronic ester of formula (IX) with an acid of formula (V), to form the compound of formula (X):

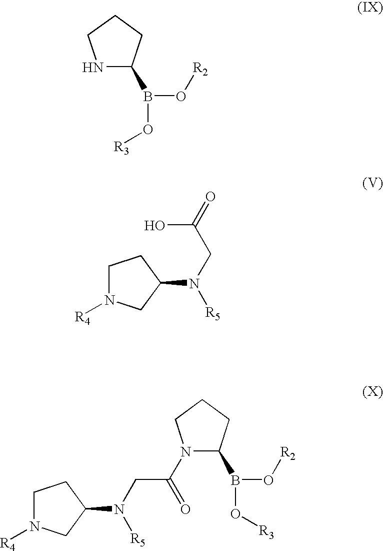

wherein R2, R3, R4 and R5 are protecting groups,

(b) removing the R4 and R5 groups from the compound of formula (X) to form the compound of formula (XI);

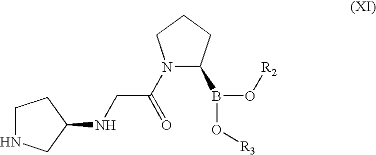

(c) reacting the compound of (XI) with a boronic acid to form the compound of formula (I) and the compound of formula (VII);

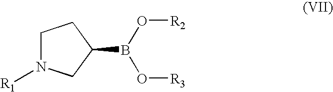

wherein R1 is a protecting group;

(d) removing the R1 group from the compound of formula (VII) to form the compound of formula (IX); and

(e) recycling the compound of formula (IX) for use in reacting step (a).

In other embodiments, the present invention relates to a method for preparing the compound of formula (I), or a pharmaceutically acceptable salt thereof, wherein the method comprises:

reacting the compound of (XI) with a boronic acid to form the compound of formula (I) and optionally the compound of formula (VII):

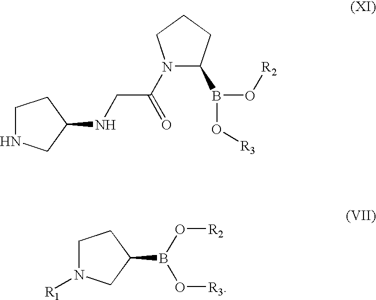

In other embodiments, the present invention relates to a method for preparing the compound of formula (I), or a pharmaceutically acceptable salt thereof, wherein the method comprises converting (e.g., by asymmetric deprotonation as described herein) the compound of formula (VI) to the compound of formula (VII) and/or the compound of formula (VIII);

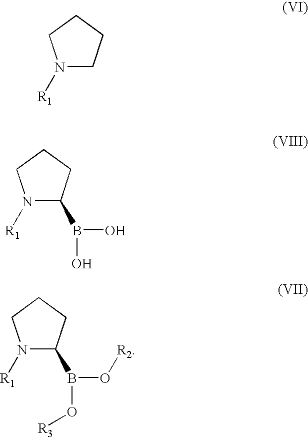

In other embodiments, the present invention relates to a pyrrolidine compound represented by formula (I) that is produced by the process described herein.

In other embodiments, the present invention relates to a pharmaceutical composition comprising a pharmaceutically acceptable carrier and a compound of formula (I) prepared by the methods described herein.

### BRIEF DESCRIPTION OF THE FIGURES

FIG. 1 is a schematic that depicts an example process for preparing the compound of formula (I).

FIG. 2 is a schematic that depicts an example process for preparing the compound of formula (I).

FIG. 3 is a schematic that depicts an example process for preparing the compound of formula (I).

FIG. 4 is a schematic that depicts an example process for preparing the compound of formula (I).

FIG. 5 illustrates the thermogravimetric analysis discussed in Example 6.

FIG. 6 illustrates the X-Ray diffractogram discussed in Example 6.

FIG. 7 illustrates the thermogravimetric analysis discussed in Example 6.

FIG. 8 illustrates the X-Ray diffractogram discussed in Example 6.

### DETAILED DESCRIPTION OF THE INVENTION

### Definitions

As used herein, unless otherwise indicated, the terms “the compound of formula (I)”, “the DPP-IV inhibitor of formula (I)”, “dutogliptin”, “active pharmaceutical ingredient” and “API” are used synonymously to refer to the compound depicted in formula (I).

Unless otherwise indicated, the terms “enantiomerically enriched” and “enantiomerically pure,” when used to describe a compound of a particular structural formula (e.g., the compounds of formulas (I), (II), (III), (IV), (V), (VI), (VII), (VIII), (IX), (X), (XI) and/or (XII)), mean greater than 50% of the enantiomer depicted in the structural formula, e.g., greater than 60%, greater than 70%, greater than 80%, greater than 90%, greater than 95%, greater than 98%, greater than 99%, greater than 99.5%, or even greater than 99.9% of the enantiomer depicted in the formula, relative to other enantiomers.

As used herein, unless otherwise indicated, “R2” and “R3” are defined as being any suitable protecting groups. In some embodiments, for example, R2 and R3 are independently selected from any alkyl, heteroatom-containing alkyl, cycloalkane, heterocycle group. In some embodiments, R2 and R3 collectively form any cyclic or heterocyclic structure with each other and/or with one or more —B—O— groups of the compound formula (VII), wherein any alkyl group, heteroatom-containing alkyl group, cyclic and/or any heterocyclic group of R2 and/or R3 is optionally substituted by one or more alkyl, cycloalkyl, alkylamino, dialkylamino, arylamino, diarylamino, amido, alkylamido, aryl, arylalkyl, heteroaryl, heteroarylalkyl, heterocycle, heterocycloalkyl, alkoxy, aryloxy, heteroaryloxy, cycloalkyloxy, cycloalkylalkyloxy, arylalkyloxy, heteroarylalkyloxy, alkoxycarbonyl, aryloxycarbonyl and/or heteroaryloxycarbonyl groups. In some embodiments, R2 and R3 are the same. In some embodiments, R2 and R3 are different. In some embodiments, R2 and R3 collectively form:

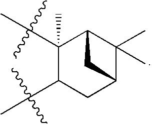

As used herein, unless otherwise indicated, “R4” and “R5” are defined as being any suitable protecting groups, for example, any suitable nitrogen protecting group or any suitable amine protecting group. In some embodiments, R4 and R5 are independently selected from benzyl carbamate (CBz), trifluoro acetate (TFA), benzyl (Bn) and t-butyl carbamate (boc) protecting groups. In some embodiments, R4 and/or R5 is CBz. In some embodiments, R4 and/or R5 is TFA. In some embodiments, R4 and/or R5 is Bn. In some embodiments, R4 and/or R5 is a boc protecting group.

As used herein, unless otherwise indicated, “R1” is defined as being any suitable protecting group. In some embodiments, R1 is a carbamate-containing protecting group or an amide-containing protecting group. In some embodiments, R1 is a carbamate-containing protecting group. In some embodiments, R1 is an amide-containing protecting group. In some embodiments, R1 is t-butyl carbamate. In some embodiments, R1 is CBz.

As used herein, unless otherwise indicated, “therapeutically effective amount”, “effective amount”, and variants thereof, mean the amount of a compound of formula (I) or a pharmaceutically acceptable salt thereof (e.g., tartrate salt) that, when administered to a mammal (e.g., human) for treating a state, disease, disorder or condition, is sufficient to affect a treatment. The “therapeutically effective amount” will vary depending on the compound, the disease and its severity and the age, sex, weight, physical condition and responsiveness of the mammal (e.g., human) to be treated. For example, a therapeutically effective amount of the compound of formula (I), or its pharmaceutically acceptable salt or hydrate, can be an amount effective to inhibit DPP-IV and/or an amount effective to treat diabetes mellitus and related conditions and/or diabetic complications.

As used herein, unless otherwise indicated, the term “treat”, in all its verb forms, is used herein to mean to relieve, alleviate, delay, manage, reduce, reverse, improve, or prevent at least one symptom of a condition, disease, or disorder in a subject, for example diabetes mellitus and related conditions and/or diabetic complications. Within the meaning of the present invention, the term “treat” also denotes, to arrest, delay the onset (i.e., the period prior to clinical manifestation of a disease) and/or reduce the risk of developing or worsening a condition, disease, or disorder in a subject, for example, diabetes mellitus and related conditions and/or diabetic complications. The term “treatment” means the act of “treating” as defined above.

The term “diabetes mellitus and related conditions” as used herein, unless otherwise indicated, refers to, but is not limited to, Type 1 diabetes, Type 2 diabetes, gestational diabetes, Maturity Onset Diabetes of the Young (MODY), impaired glucose tolerance, impaired fasting glucose, hyperglycemia, impaired glucose metabolism, insulin resistance, obesity, diabetic complications, and the like. The term “diabetic complications”, unless otherwise indicated, refers to but is not limited to conditions, disorders, and/or maladies associated with diabetes, e.g., retinopathies, neuropathies, nephropathies, cardiomyopathies, dermopathies, arthrosclerosis, coronary artery disease and/or other known complications associated with diabetes.

As used herein, unless otherwise indicated, the terms “DPP-VII, DPP-VIII, DPP-IX and FAP” mean amino dipeptidyl peptidase VII, VIII, IX and fibroblast activation protein, respectively. The term “DPP-IV” denotes dipeptidyl peptidase IV (EC 3.4.14.5; DPP-IV), also known as “CD-26.”

As used herein, unless otherwise indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used herein, unless otherwise indicated, the terms “pharmaceutical salt”, “pharmaceutically acceptable salt”, and variants thereof, refer to any pharmaceutically acceptable salt of dutogliptin, for example, any salt with an inorganic base, organic base (e.g., basic amino acids, for example, arginine, lysine or ornithine), inorganic acid, and/or organic acid (e.g., acidic amino acids, for example, aspartic acid or glutamic acid). Suitable inorganic bases include, but are not limited to, alkali metals (e.g., lithium, sodium or potassium), alkaline earth metals (e.g., calcium, magnesium or aluminum). Suitable inorganic acids include, but are not limited to, hydro-halogen acids, hydrochloric acid, hydroboric acid, nitric acid, sulfuric acid and/or phosphoric acid. Suitable organic acids include, but are not limited to, mono-, di- and tri-carboxylic or sulfonic acids of 1 to 20 carbons, optionally containing 1 to 6 hydroxyl groups. Further examples of pharmaceutically acceptable salts are readily known to those of ordinary skill in the art, for example, as described in the Journal of Medicinal Chemistry, 50, 6665 (2007), the contents of which are incorporated herein by reference.

As used herein, unless otherwise indicated, the term “stereoisomer” refers to one of the absolute configurations of a single organic molecule having at least one asymmetric carbon. Included within the definition of a stereoisomer are enantiomers and diastereomers. As used herein, unless otherwise indicated, the term “enantiomer” refers to any member of a pair of stereoisomers having the same molecular structure and at least one asymmetric carbon such that the stereoisomers of the pair are non-superimposable mirror images of each other.

As used herein, unless otherwise indicated, the term “prodrug” refers to a pharmaceutically acceptable compound that will convert to the active ingredient or an active metabolite thereof upon administration of the prodrug to a living organism, preferably a mammal, more preferably a human. The conversion may occur by enzymatic action, chemical hydrolysis, oxidation, reduction or any other in vivo physiological process for chemical or biochemical reaction.

As used herein, unless otherwise indicated, the term “solvate” refers to a solid, crystalline form of a compound which also incorporates molecules of a solvent into the crystal structure. Organic solvents as well as water are included. Another description of a water solvate is a “hydrate” or “hydrated form”.

The term “tartrate” is used herein to refer to a salt of tartaric acid. The tartaric acid can be of any stereochemical configuration, e.g., a salt of D-tartaric acid, L-tartaric acid, DL-tartaric acid, meso-tartaric acid, or any combination or mixture thereof.

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

The term “consisting essentially of”, and variants thereof, when used to refer to a pharmaceutical composition or formulation, are used herein to mean that the composition or formulation includes the compound of formula (I) and other desired pharmaceutically inactive additives, excipients, and/or components, and but no other active pharmaceutical ingredient(s).

### DETAILED DESCRIPTION

Methods are provided for preparing the compound of formula (I)

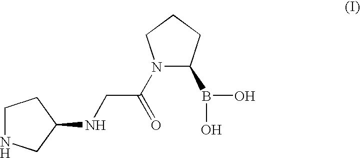

or a pharmaceutically acceptable salt thereof (e.g., tartrate or citrate salt thereof), a prodrug thereof, a solvate thereof, a hydrate thereof, and/or an enantiomer thereof, e.g., wherein the compound of formula (I) is a DPP-IV inhibitor.

In some embodiments, the method comprises: (a) reacting or coupling a boronic ester of formula (IX) with an acid of formula (V), to form the compound of formula (X):

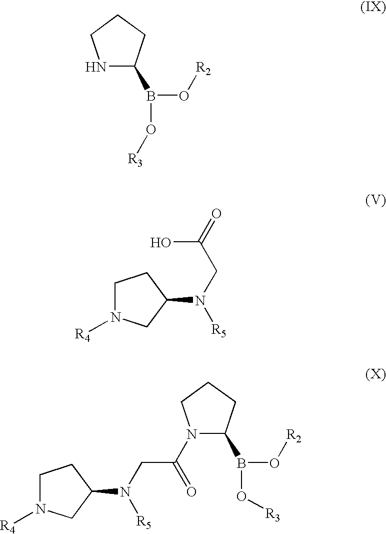

wherein R2, R3, R4, and R5 are protecting groups,

(b) removing the R4 and R5 groups from the compound of formula (X) to form the compound of formula (XI);

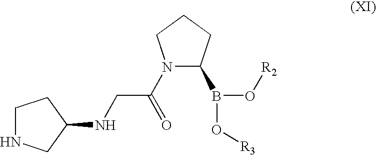

(c) reacting the compound of (XI) with a boronic acid to form the compound of formula (I) and optionally the compound of formula (VII),

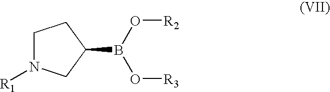

wherein R1 is a protecting group;

(d) optionally, if any compound of formula (VII) is formed in reacting step (c), removing the R1 group from the compound of formula (VII) to form the compound of formula (IX); and

(e) optionally recycling the compound of formula (IX) for use in reacting step (a).

In some embodiments, the method comprises: (a) coupling a boronic ester of formula (IX) with an acid of formula (V), to form the compound of formula (X), wherein R2, R3, R4, and R5 are protecting groups;

(b) removing the R4 and R5 groups from the compound of formula (X) to form the compound of formula (XI);

(c) reacting the compound of (XI) with a boronic acid to form the compound of formula (I) and the compound of formula (VII), wherein R1 is any suitable protecting group;

(d) removing the R1 group from the compound of formula (VII) to form the compound of formula (IX); and

(e) recycling the compound of formula (IX) for use in reacting step (a).

In some embodiments, the method comprises reacting or coupling a boronic ester of formula (IX) with the acid of formula (V) to form the compound of formula (X).

In some embodiments, the method comprises reacting or converting the compound of (XI) with a boronic acid (e.g., the compound of formula (VIII), for example in enantiomerically enriched form) to form the compound of formula (I) and optionally the compound of formula (VII).

In some embodiments, the method comprises (a) reacting or coupling a boronic ester of formula (IX) with the acid of formula (V) to form the compound of formula (X); (b) reacting the compound of (XI) with a boronic acid (e.g., the compound of formula (VIII), for example in enantiomerically enriched form) to form the compound of formula (I) and optionally the compound of formula (VII); (c) converting the compound of formula (VII) to the compound of formula (IX); and optionally (d) recycling the compound of formula (VII) for use in coupling step (a).

### Reacting Step (a):

In some embodiments, reacting step (a) comprises coupling or reacting a boronic ester of formula (IX) with an acid of formula (V), to form the compound of formula (X). In some embodiments, reacting step (a) comprises coupling or reacting the boronic ester of formula (IX) and the acid of formula (V) under suitable amide coupling conditions. In some embodiments, reacting step (a) comprises coupling or reacting the boronic ester of formula (IX) with the acid of formula (V) (e.g., with an activated form of the acid of formula (V)) with an anhydride (e.g., methylchloroformate, ethylchloroformate, isobutylchloroformate, etc. . . . ), a carbodiimide (e.g., N,N′-dicyclohexylcarbodiimide (DCC) or 1-ethyl-3-(3-dimethylaminopropyl) carbodiimide (EDC) and/or an acid halide (e.g., acid chloride or acid bromide). In some embodiments, reacting step (a) comprises coupling the boronic ester of formula (IX) and the acid of formula (V) using, or in the presence of, one or more reagents suitable for activating the carboxylic acid of the boronic ester of formula (IX). In some embodiments, reacting step (a) comprises coupling or reacting the boronic ester of formula (IX) with an anhydride (e.g., mixed anhydride) of the acid of formula (V). In some embodiments, reacting step (a) comprises activating the acid of formula (V) (e.g., with a carbodiimide) and reacting the activated acid of formula (V) with the boronic ester of formula (IX). In some embodiments, reacting step (a) comprises coupling or reacting the boronic ester of formula (IX) and the acid chloride of formula (V).

In some embodiments, the compound of formula (X) formed in reacting step (a) is enantiomerically enriched. In some embodiments, the compound of formula (X) formed in reacting step (a) comprises greater than 50% of the R enantiomer. In some embodiments, the compound of formula (X) formed in reacting step (a) comprises greater than 60% of the R enantiomer. In some embodiments, the compound of formula (X) formed in reacting step (a) comprises greater than 70% of the R enantiomer. In some embodiments, the compound of formula (X) formed in reacting step (a) comprises greater than 80% of the R enantiomer. In some embodiments, the compound of formula (X) formed in reacting step (a) comprises greater than 90% of the R enantiomer. In some embodiments, the compound of formula (X) formed in reacting step (a) comprises greater than 95% of the R enantiomer. In some embodiments, the compound of formula (X) formed in reacting step (a) comprises greater than 99% of the R enantiomer. In some embodiments, the compound of formula (X) formed in reacting step (a) comprises less than 50% of the S enantiomer. In some embodiments, the compound of formula (X) formed in reacting step (a) comprises less than 40% of the S enantiomer. In some embodiments, the compound of formula (X) formed in reacting step (a) comprises less than 30% of the S enantiomer. In some embodiments, the compound of formula (X) formed in reacting step (a) comprises less than 20% of the S enantiomer. In some embodiments, the compound of formula (X) formed in reacting step (a) comprises less than 10% of the S enantiomer. In some embodiments, the compound of formula (X) formed in reacting step (a) comprises less than 5% of the S enantiomer. In some embodiments, the compound of formula (X) formed in reacting step (a) comprises less than 1% of the S enantiomer.

In some embodiments, the compound of formula (IX) used in reacting step (a) is enantiomerically enriched. In some embodiments, the compound of formula (IX) used in reacting step (a) comprises greater than 50% of the R enantiomer. In some embodiments, the compound of formula (IX) used in reacting step (a) comprises greater than 60% of the R enantiomer. In some embodiments, the compound of formula (IX) used in reacting step (a) comprises greater than 70% of the R enantiomer. In some embodiments, the compound of formula (IX) used in reacting step (a) comprises greater than 80% of the R enantiomer. In some embodiments, the compound of formula (IX) used in reacting step (a) comprises greater than 90% of the R enantiomer. In some embodiments, the compound of formula (IX) used in reacting step (a) comprises greater than 95% of the R enantiomer. In some embodiments, the compound of formula (IX) used in reacting step (a) comprises greater than 99% of the R enantiomer. In some embodiments, the compound of formula (IX) used in reacting step (a) comprises less than 50% of the S enantiomer. In some embodiments, the compound of formula (IX) used in reacting step (a) comprises less than 40% of the S enantiomer. In some embodiments, the compound of formula (IX) used in reacting step (a) comprises less than 30% of the S enantiomer. In some embodiments, the compound of formula (IX) used in reacting step (a) comprises less than 20% of the S enantiomer. In some embodiments, the compound of formula (IX) used in reacting step (a) comprises less than 10% of the S enantiomer. In some embodiments, the compound of formula (IX) used in reacting step (a) comprises less than 5% of the S enantiomer. In some embodiments, the compound of formula (IX) used in reacting step (a) comprises less than 1% of the S enantiomer.

### Removing Step (b):

In some embodiments, removing step (b) comprises removing or deprotecting the R4 and/or R5 groups from the compound of formula (X) to form the compound of formula (XI). The particular method used in removing step (b) depends on the type of protecting group that exists at the R4 and R5 positions. For example, in some embodiments, R4 and/or R5 is t-butyl carbamate (boc), and removing step (b) comprises subjecting the compound of formula (X) to acidic conditions (e.g., anhydrous acidic conditions or aqueous acidic conditions) Any suitable acid can be used in this regard. In some embodiments, the acid is hydrochloric acid. In some embodiments, the acid is trifluoroacetatic acid. In some embodiments, R4 and/or R5 is benzyl carbamate (CBz), and removing step (b) comprises subjecting the compound of formula (X) to hydrogenation conditions. In some embodiments, R4 and/or R5 is CBz, and removing step (b) comprises reacting or mixing the compound of formula (X) with a catalyst (e.g., a palladium catalyst or a platinum catalyst), for example, in the presence of hydrogen. In some embodiments, R4 and/or R5 is CBz, and removing step (b) comprises reacting or mixing the compound of formula (X) with a catalyst (e.g., a palladium catalyst or a platinum catalyst), in combination with hydrogen or hydrogen source such as ammonium formate, formic acid, cyclohexadiene or cyclohexene. In some embodiments, R4 and/or R5 is CBz, and removing step (b) comprises reacting or mixing the compound of formula (X) with a catalyst (e.g., a palladium catalyst or a platinum catalyst) and hydrogen. In some embodiments, R4 and/or R5 is CBz, and removing step (b) comprises reacting or mixing the compound of formula (X) with a catalyst (e.g., a palladium catalyst or a platinum catalyst) and a formate compound (e.g., ammonium formate). In some embodiments, R4 and/or R5 is CBz, and removing step (b) comprises reacting or mixing the compound of formula (X) with a catalyst (e.g., a palladium catalyst or a platinum catalyst) and formic acid. In some embodiments, R4 and/or R5 is CBz, and removing step (b) comprises reacting or mixing the compound of formula (X) with a catalyst (e.g., a palladium catalyst or a platinum catalyst) and cyclohexadiene. In some embodiments, R4 and/or R5 is TFA, and removing step (b) comprises subjecting the compound of formula (X) to alkaline hydrolysis conditions. In some embodiments, R4 and/or R5 is TFA and removing step (b) comprises mixing the boronic acid of formula (VIII) with a carbonate and an alcohol (e.g., methanol). In some embodiments, R4 and/or R5 is TFA and removing step (b) comprising subjecting the compound of formula (X) to basic condition, e.g., by mixing the compound with a base (for example, a base selected from ammonia, hydroxide, carbonate, and alcohol). In some embodiments, R4 and/or R5 is Bn and removing step (b) comprising subjecting the compound of formula (X) to hydrogenation conditions. In some embodiments, R4 and/or R5 is Bn, and removing step (b) comprises reacting or mixing the compound of formula (X) with a catalyst (e.g., a palladium catalyst or a platinum catalyst), for example, in the presence of hydrogen.

In some embodiments, the compound of formula (XI) formed in removing step (b) is enantiomerically enriched. In some embodiments, the compound of formula (XI) formed in removing step (b) comprises greater than 50% of the R enantiomer (e.g., R/R enantiomer). In some embodiments, the compound of formula (XI) formed in removing step (b) comprises greater than 60% of the R enantiomer (e.g., R/R enantiomer). In some embodiments, the compound of formula (XI) formed in removing step (b) comprises greater than 70% of the R enantiomer (e.g., R/R enantiomer). In some embodiments, the compound of formula (XI) formed in removing step (b) comprises greater than 80% of the R enantiomer (e.g., R/R enantiomer). In some embodiments, the compound of formula (XI) formed in removing step (b) comprises greater than 90% of the R enantiomer (e.g., R/R enantiomer). In some embodiments, the compound of formula (XI) formed in removing step (b) comprises greater than 95% of the R enantiomer (e.g., R/R enantiomer). In some embodiments, the compound of formula (XI) formed in removing step (b) comprises greater than 99% of the R enantiomer (e.g., RJR enantiomer). In some embodiments, the compound of formula (XI) formed in removing step (b) comprises less than 50% of the S enantiomer (e.g., S/S, S/R, or R/S enantiomer). In some embodiments, the compound of formula (XI) formed in removing step (b) comprises less than 40% of the S enantiomer (e.g., S/S, S/R, or R/S enantiomer). In some embodiments, the compound of formula (XI) formed in removing step (b) comprises less than 30% of the S enantiomer (e.g., S/S, S/R, or R/S enantiomer). In some embodiments, the compound of formula (XI) formed in removing step (b) comprises less than 20% of the S enantiomer (e.g., S/S, S/R, or R/S enantiomer). In some embodiments, the compound of formula (XI) formed in removing step (b) comprises less than 10% of the S enantiomer (e.g., S/S, S/R, or R/S enantiomer). In some embodiments, the compound of formula (XI) formed in removing step (b) comprises less than 5% of the S enantiomer (e.g., S/S, S/R, or R/S enantiomer). In some embodiments, the compound of formula (XI) formed in removing step (b) comprises less than 1% of the S enantiomer (e.g., S/S, S/R, or R/S enantiomer).

### Reacting Step (c):

In some embodiments, reacting step (c) comprises reacting or converting the compound of (XI) with a boronic acid to form the compound of formula (I) and optionally the compound of formula (VII). In some embodiments, both the compound of formula (I) and the compound of formula (VII) are formed by reacting step (c).

Reacting step (c) can be performed using any suitable boronic acid. In some embodiments, the boronic acid is a phenylboronic acid. In some embodiments, the boronic acid is an enantiomerically enriched form of a boronic acid. In some embodiments, the boronic acid used in reacting step (c) is a non-racemic boronic acid. In some embodiments, the boronic acid used in reacting step (c) is an enantiomerically enriched form of the compound of formula (VIII):

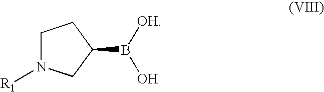

In some embodiments, the boronic acid of formula (VIII) used by reacting step (c) comprises greater than 50% of the R enantiomer. In some embodiments, the compound of formula (VIII) used by reacting step (c) comprises greater than 60% of the R enantiomer. In some embodiments, the compound of formula (VIII) used by reacting step (c) comprises greater than 70% of the R enantiomer. In some embodiments, the compound of formula (VIII) used by reacting step (c) comprises greater than 80% of the R enantiomer. In some embodiments, the compound of formula (VIII) used by reacting step (c) comprises greater than 90% of the R enantiomer. In some embodiments, the compound of formula (VIII) used by reacting step (c) comprises greater than 95% of the R enantiomer. In some embodiments, the compound of formula (VIII) used by reacting step (c) comprises greater than 99% of the R enantiomer. In some embodiments, the compound of formula (VIII) used by reacting step (c) comprises less than 50% of the S enantiomer. In some embodiments, the compound of formula (VIII) used by reacting step (c) comprises less than 40% of the S enantiomer. In some embodiments, the compound of formula (VIII) used by reacting step (c) comprises less than 30% of the S enantiomer. In some embodiments, the compound of formula (VIII) used by reacting step (c) comprises less than 20% of the S enantiomer. In some embodiments, the compound of formula (VIII) used by reacting step (c) comprises less than 10% of the S enantiomer. In some embodiments, the compound of formula (VIII) used by reacting step (c) comprises less than 5% of the S enantiomer. In some embodiments, the compound of formula (VIII) used by reacting step (c) comprises less than 1% of the S enantiomer.

In some embodiments, the boronic acid used in reacting step (c) is a racemate represented by the formula (VIIIa):

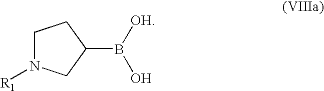

The reacting step (c) can be performed with any suitable additional reagents and/or reactants, as well as under any suitable reaction conditions. In some embodiments, reacting step (c) comprises reacting the compound of (XI) with a boronic acid in a biphasic system or solvent (e.g., comprising aqueous and organic or anhydrous components). In some embodiments, reacting step (c) comprises reacting the compound of (XI) with a boronic acid in the presence of an acid (e.g., tartaric acid or citric acid). In some embodiments, reacting step (c) comprises reacting the compound of (XI) with a boronic acid in a biphasic solvent system and in the presence of an acid (e.g., tartaric acid or citric acid). In some embodiments, reacting step (c) comprises subjecting the compound of (XI) to acidic conditions.

In some embodiments, reacting step (c) produces an enantiomerically enriched form of the compound of formula (I) and an enantiomerically enriched form of the compound of formula (VII).

### Removing Step (d) and Recycling Step (e)

In some embodiments, the method comprises removing or deprotecting the R1 group from the compound of formula (VII) to form the compound of formula (IX) and recycling the compound of formula (IX) for use in reacting step (a).

The particular method used in removing step (d) depends on the type of protecting group that exists at the R1 position. For example, in some embodiments, R1 is t-butyl carbamate (boc), and removing step (d) comprises subjecting the compound of formula (VII) to acidic conditions (e.g., anhydrous acidic conditions or aqueous acidic conditions) Any suitable acid can be used in this regard, for example, hydrochloric acid. In some embodiments, R1 is t-butyl carbamate, and removing step (d) comprises reacting the compound of formula (VII) with hydrochloric acid. In some embodiments, R1 is benzyl carbamate (CBz), and removing step (d) comprises subjecting the compound of formula (VII) to hydrogenation conditions. In some embodiments, R1 is CBz, and removing step (d) comprises reacting or mixing the compound of formula (VII) with a catalyst (e.g., a palladium catalyst or a platinum catalyst), for example, in the presence of hydrogen. In some embodiments, R1 is CBz, and removing step (d) comprises reacting or mixing the compound of formula (VII) with a catalyst (e.g., a palladium catalyst or a platinum catalyst), in combination with hydrogen or a source of hydrogen (for example ammonium formate, formic acid, cyclohexyldiene and/or cyclohexane). In some embodiments, R1 is CBz, and removing step (d) comprises reacting or mixing the compound of formula (VII) with a catalyst (e.g., a palladium catalyst or a platinum catalyst) and hydrogen. In some embodiments, R1 is CBz, and removing step (d) comprises reacting or mixing the compound of formula (VII) with a catalyst (e.g., a palladium catalyst or a platinum catalyst) and a formate compound (e.g., ammonium formate). In some embodiments, R1 is CBz, and removing step (d) comprises reacting or mixing the compound of formula (VII) with a catalyst (e.g., a palladium catalyst or a platinum catalyst) and formic acid. In some embodiments, R1 is CBz, and removing step (d) comprises reacting or mixing the compound of formula (VII) with a catalyst (e.g., a palladium catalyst or a platinum catalyst) and cyclohexyldiene. In some embodiments, R1 is TFA, and removing step (d) comprises subjecting the compound of formula (VII) to alkaline hydrolysis conditions. In some embodiments, R1 is TFA and removing step (d) comprises mixing the compound of formula (VII) with a carbonate and an alcohol (e.g., methanol). In some embodiments, R1 is TFA and removing step (d) comprising subjecting the compound of formula (VII) to basic condition, e.g., by mixing the compound with a base (for example, a base selected from ammonia, hydroxide, carbonate, and alcohol).

In some embodiments, the compound of formula (IX) formed by removing step (d) is enantiomerically enriched. In some embodiments, the compound of formula (IX) formed by removing step (d) comprises greater than 50% of the R enantiomer. In some embodiments, the compound of formula (IX) formed by removing step (d) comprises greater than 60% of the R enantiomer. In some embodiments, the compound of formula (IX) formed by removing step (d) comprises greater than 70% of the R enantiomer. In some embodiments, the compound of formula (IX) formed by removing step (d) comprises greater than 80% of the R enantiomer. In some embodiments, the compound of formula (IX) formed by removing step (d) comprises greater than 90% of the R enantiomer. In some embodiments, the compound of formula (IX) formed by removing step (d) comprises greater than 95% of the R enantiomer. In some embodiments, the compound of formula (IX) formed by removing step (d) comprises greater than 99% of the R enantiomer. In some embodiments, the compound of formula (IX) formed by removing step (d) comprises less than 50% of the S enantiomer. In some embodiments, the compound of formula (IX) formed by removing step (d) comprises less than 40% of the S enantiomer. In some embodiments, the compound of formula (IX) formed by removing step (d) comprises less than 30% of the S enantiomer. In some embodiments, the compound of formula (IX) formed by removing step (d) comprises less than 20% of the S enantiomer. In some embodiments, the compound of formula (IX) formed by removing step (d) comprises less than 10% of the S enantiomer. In some embodiments, the compound of formula (IX) formed by removing step (d) comprises less than 5% of the S enantiomer. In some embodiments, the compound of formula (IX) formed by removing step (d) comprises less than 1% of the S enantiomer.

The compound of formula (IX) produced by removing step (d) can be recycled (e.g., in recycling step (e)) for use in reacting step (a). In some embodiments, the method requires no processing steps (e.g., separation steps, for example, enantiomer separation steps, crystallization, fractional crystallization, and/or purification steps) other than removing step (d) for producing an enantiomerically enriched form of the compound of formula (IX) prior to the recycling step, i.e., the method requires no intervening processing steps between removing step (d) and recycling step (e). In some embodiments, a recrystallization step is performed after removing step (d) to enhance the purity of the enantiomerically enriched form of the compound of formula (IX) prior to recycling. In some embodiments, minimal or no undesired enantiomers (e.g., S enantiomer of the compound of formula (IX)) are produced by removing (d) and, therefore, no stripping or recapture process steps are required for recapturing or removing desired components (e.g., pinanediol) from the undesired enantiomer. In some embodiments, some undesired enantiomer (e.g., S enantiomer of the compound of formula (IX)) and the method further comprises: (i) separating the desired enantiomer from the undesired enantiomer through crystallization; and optionally (ii) recapturing desired component(s) (e.g., pinanediol) from the undesired enantiomer.

### Preparation of the Boronic Ester of Formula (IX)

In some embodiments, the boronic ester of formula (IX) used in reacting step (a) is prepared by:

(i) converting the compound of formula (VI) to the compound of formula (VII) and/or the compound of formula (VIII) in any suitable manner;

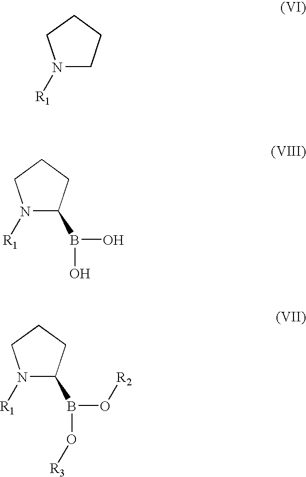

(ii) optionally, if any compound of formula (VIII) is produced in step (i), converting the compound of formula (VIII) to the compound of formula (VII); and

(iii) deprotecting the R1 group of the compound of formula (VII) to form the boronic ester of formula (IX).

In some embodiments, converting step (i) comprises reacting the compound of formula (VI) with a chiral ligand (e.g., a chiral amine), a base (e.g., an alkyl lithium base, for example, sec-butyllithium), and subsequently with a borate (e.g., a boronic ester), to form the compound of formula (VII) and/or the compound of formula (VIII), wherein R1 of the compound of formula (VI) is any suitable protecting group as discussed above. In some embodiments, the converting step comprises deprotonating (e.g., asymmetrically deprotonating) the compound of formula (VI) with a chiral ligand (e.g., a chiral amine) and a base (e.g., sec-butyllithium), and capturing the resulting anion with a borate (e.g., a boronic ester, for example trimethyl borate, triisopropyl borate, or the borate of formula (XIV)) to form the compound of formula (VII) and/or the compound of formula (VIII).

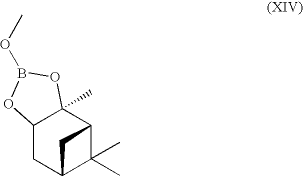

Any suitable chiral ligand can be used in the present method. In some embodiments, the chiral ligand is a chiral amine ligand for example a chiral diamine ligand. Any suitable chiral diamine ligand can be used in this regard. In some preferred embodiments, the chiral amine ligand used in the context of the present invention is ((1S,2S)-dimethyl-bis(3,3-dimethyl butyl) cyclohexane-1,2-diamine). In some embodiments, the chiral amine ligand is selected from one or more of the following example ligands:

### (1S,2S)—N,N′-Bis-(3,3-dimethyl-butyl)-N,N′-dimethyl-cyclohexane-1,2-diamine

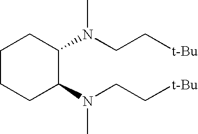

### (1R,2R)—N,N′-Bis-(3,3-dimethyl-butyl)-N,N′-dimethyl-cyclohexane-1,2-diamine

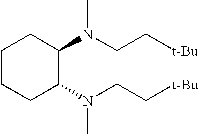

### (−)-sparteine

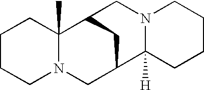

### (1R,2S,9S)-11-methyl-7,11-diazatricyclo[7.3.1.0]tridecane

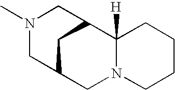

### (1S,2S)—N,N′-Bis-(2,2-dimethyl-propyl)-N,N′-dimethyl-cyclohexane-1,2-diamine

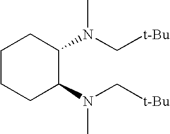

### (1R,2R)—N,N′-Bis-(2,2-dimethyl-propyl)-N,N′-dimethyl-cyclohexane-1,2-diamine

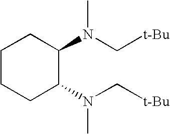

### (1S,2S)—N,N′-Diisopropyl-N,N′-dimethyl-cyclohexane-1,2-diamine

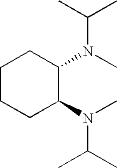

### (1R,2R)—N,N′-Diisopropyl-N,N′-dimethyl-cyclohexane-1,2-diamine

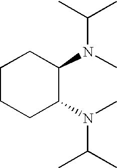

### R)-1-Methyl-2-pyrrolidin-1-ylmethyl-pyrrolidine

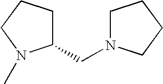

### (S)-1-Methyl-2-pyrrolidin-1-ylmethyl-pyrrolidine

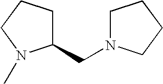

### [(S)-1-((S)-1-Methyl-pyrrolidin-2-ylmethyl)-pyrrolidin-2-yl]-methanol

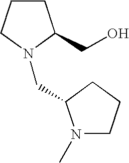

### [(R)-1-((R)-1-Methyl-pyrrolidin-2-ylmethyl)-pyrrolidin-2-yl]-Methanol

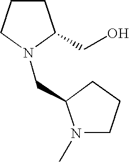

### Pyrrolidinylboronates

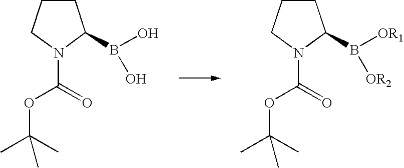

In some embodiments, Ligand-Directed Asymmetric Synthesis of Pyrrolidinylboronic acid occurs as follows:

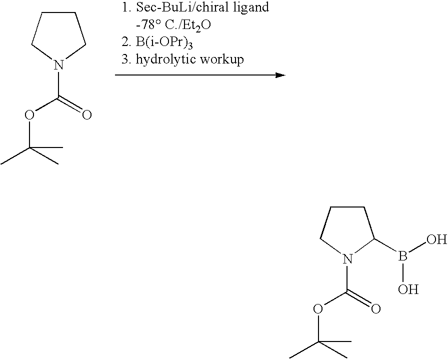

In some embodiments, converting step (ii) comprises reacting the compound of formula (VIII) with an alcohol or diol compound (e.g., pinanediol or pinnacol diol) to form the compound of formula (VII).

In some embodiments, deprotecting step (iii) comprises removing or deprotecting the R1 group of the compound of formula (VII) in any manner discussed herein, to form the boronic ester of formula (IX).

In some preferred embodiments, the compound of formula (VII) and/or the compound of formula (VIII) formed in steps (i)-(ii) are in enantiomerically enriched form. In some embodiments, the compound of formula (VII) formed in steps (i)-(ii) comprises greater than 50% of the R enantiomer. In some embodiments, the compound of formula (VII) formed in steps (i)-(ii) comprises greater than 60% of the R enantiomer. In some embodiments, the compound of formula (VII) formed in steps (i)-(ii) comprises greater than 70% of the R enantiomer. In some embodiments, the compound of formula (VII) formed in steps (i)-(ii) comprises greater than 80% of the R enantiomer. In some embodiments, the compound of formula (VII) formed in steps (i)-(ii) comprises greater than 90% of the R enantiomer. In some embodiments, the compound of formula (VII) formed in steps (i)-(ii) comprises greater than 95% of the R enantiomer. In some embodiments, the compound of formula (VII) formed in steps (i)-(ii) comprises greater than 99% of the R enantiomer. In some embodiments, the compound of formula (VII) formed in steps (i)-(ii) comprises less than 50% of the S enantiomer. In some embodiments, the compound of formula (VII) formed in steps (i)-(ii) comprises less than 40% of the S enantiomer. In some embodiments, the compound of formula (VII) formed in steps (i)-(ii) comprises less than 30% of the S enantiomer. In some embodiments, the compound of formula (VII) formed in steps (i)-(ii) comprises less than 20% of the S enantiomer. In some embodiments, the compound of formula (VII) formed in steps (i)-(ii) comprises less than 10% of the S enantiomer. In some embodiments, the compound of formula (VII) formed in steps (i)-(ii) comprises less than 5% of the S enantiomer. In some embodiments, the compound of formula (VII) formed in steps (i)-(ii) comprises less than 1% of the S enantiomer. In some embodiments, the compound of formula (VIII) formed in step (i) comprises greater than 50% of the R enantiomer. In some embodiments, the compound of formula (VIII) formed in step (i) comprises greater than 60% of the R enantiomer. In some embodiments, the compound of formula (VIII) formed in step (i) comprises greater than 70% of the R enantiomer. In some embodiments, the compound of formula (VIII) formed in step (i) comprises greater than 80% of the R enantiomer. In some embodiments, the compound of formula (VIII) formed in step (i) comprises greater than 90% of the R enantiomer. In some embodiments, the compound of formula (VIII) formed in step (i) comprises greater than 95% of the R enantiomer. In some embodiments, the compound of formula (VIII) formed in step (i) comprises greater than 99% of the R enantiomer. In some embodiments, the compound of formula (VIII) formed in step (i) comprises less than 50% of the S enantiomer. In some embodiments, the compound of formula (VIII) formed in step (i) comprises less than 40% of the S enantiomer. In some embodiments, the compound of formula (VIII) formed in step (i) comprises less than 30% of the S enantiomer. In some embodiments, the compound of formula (VIII) formed in step (i) comprises less than 20% of the S enantiomer. In some embodiments, the compound of formula (VIII) formed in step (i) comprises less than 10% of the S enantiomer. In some embodiments, the compound of formula (VIII) formed in step (i) comprises less than 5% of the S enantiomer. In some embodiments, the compound of formula (VIII) formed in step (i) comprises less than 1% of the S enantiomer.

In some preferred embodiments, the compound of formula (IX) formed in step (iii) is in enantiomerically enriched form. In some embodiments, the compound of formula (IX) formed in step (iii) comprises greater than 50% of the R enantiomer. In some embodiments, the compound of formula (IX) formed in step (iii) comprises greater than 60% of the R enantiomer. In some embodiments, the compound of formula (IX) formed in step (iii) comprises greater than 70% of the R enantiomer. In some embodiments, the compound of formula (IX) formed in step (iii) comprises greater than 80% of the R enantiomer. In some embodiments, the compound of formula (IX) formed in step (iii) comprises greater than 90% of the R enantiomer. In some embodiments, the compound of formula (IX) formed in step (iii) comprises greater than 95% of the R enantiomer. In some embodiments, the compound of formula (IX) formed in step (iii) comprises greater than 99% of the R enantiomer. In some embodiments, the compound of formula (IX) formed in step (iii) comprises less than 50% of the S enantiomer. In some embodiments, the compound of formula (IX) formed in step (iii) comprises less than 40% of the S enantiomer. In some embodiments, the compound of formula (IX) formed in step (iii) comprises less than 30% of the S enantiomer. In some embodiments, the compound of formula (IX) formed in step (iii) comprises less than 20% of the S enantiomer. In some embodiments, the compound of formula (IX) formed in step (iii) comprises less than 10% of the S enantiomer. In some embodiments, the compound of formula (IX) formed in step (iii) comprises less than 5% of the S enantiomer. In some embodiments, the compound of formula (IX) formed in step (iii) comprises less than 1% of the S enantiomer.

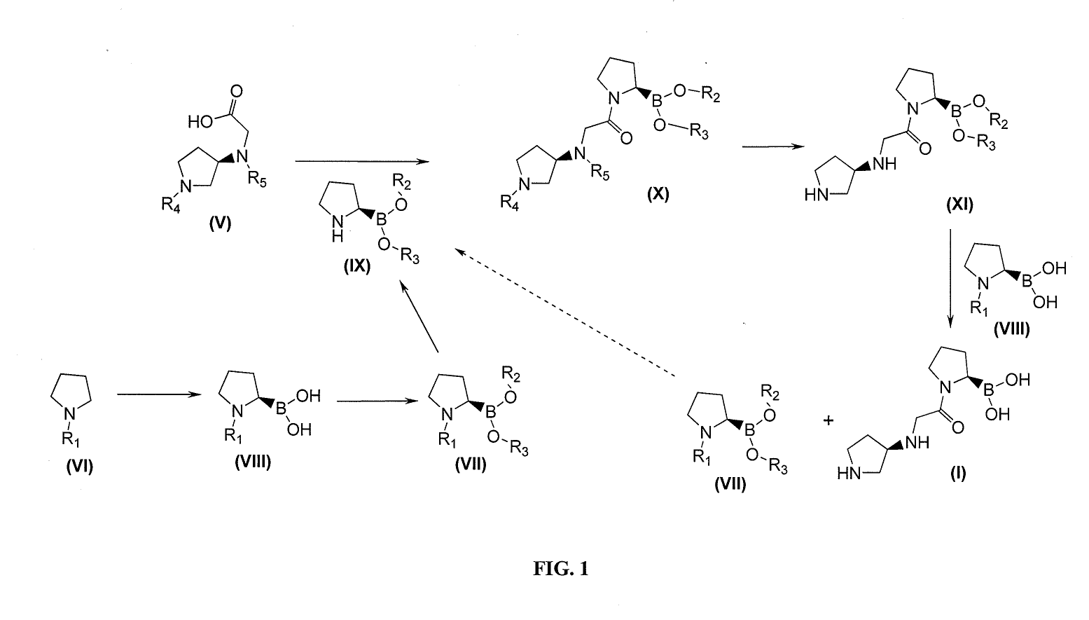

FIGS. 1-3 depict example embodiments of the method. In some embodiments, as is depicted in FIG. 1, a suitably protected pyrrolidine (VI) is asymmetrically deprotonated in the presence of an appropriate chiral ligand such as ((1S,2S)-Dimethyl-bis(3,3-dimethyl butyl) cyclohexane-1,2-diamine) and base (i.e., sec-butyl lithium) combination and the resulting anion is captured with a boronic ester such as trimethyl or triisopropyl borate to form enantiomerically enriched boronic acid (VIII). The boronic acid (VIII) is esterified to give boronic ester (VII), which is subsequently deprotected to boronic ester (IX). Coupling of boronic ester (IX) with acid (V) proceeds under general amide coupling conditions (i.e. mixed anhydrides, carbodiimides or acid chloride, etc) to provide peptide (X). Removal of the nitrogen protecting groups yields compound (XI) which is subsequently converted to boronic acid (I) by transesterification with boronic acid (VIII) or other suitable boronic acids (e.g., phenylboronic acid). The newly generated pyrrolidine (VII) can then be deprotected to generate pyrrolidine (IX) that can be recycled into the process.

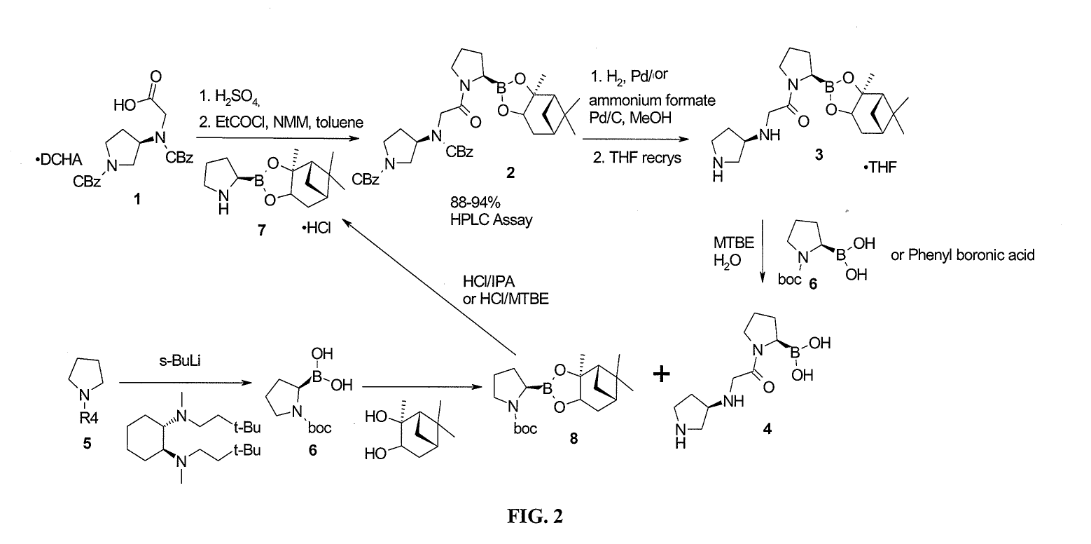

In some embodiments, as is depicted in FIG. 2, a suitably protected pyrrolidine (5) is asymmetrically deprotonated in the presence of (1S,2S)-dimethyl-bis(3,3-dimethyl butyl) cyclohexane-1,2-diamine and sec-butyl lithium and the resulting anion is captured with a boronic ester (e.g., trimethylborate) to form enantiomerically enriched boronic acid (6) (i.e., R-2-Boc-pyrrolidine boronic acid). The boronic acid (6) is esterified to give boronic ester (8) (i.e., (R)-2-((1S,2S,8S)-2,9,9-Trimethyl-3,5-dioxa-4-bora-tricyclo[6.1.1.0^{2,6}]dec-4-yl)-pyrrolidine-1-carboxylic acid tert-butyl ester), which is subsequently deprotected to form boronic ester (7) (i.e., (R)-2-((1S,2S,8S)-2,9,9-Trimethyl-3,5-dioxa-4-bora-tricyclo[6.1.1.0^{2,6}]dec-4-yl)-pyrrolidine hydrochloride). Coupling of boronic ester (7) with acid (1) (i.e., (R)-3-(benzyloxycarbonyl-carboxymethyl-amino)-pyrrolidine-1-carboxylic acid benzyl ester•DCHA salt) proceeds under general amide coupling conditions to provide peptide (2) (i.e., (R)-3-(benzyloxycarbonyl-{2-oxo-2-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethyl}-amino)-pyrrolidine-1-carboxylic acid benzyl ester). Removal of the nitrogen protecting groups yields compound (3) (i.e., 2-((R)-Pyrrolidin-3-ylamino)-1-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethanone) which is subsequently converted to dutogliptin (4) by transesterification with boronic acid (6) (i.e., R-2-Boc-pyrrolidine boronic acid)

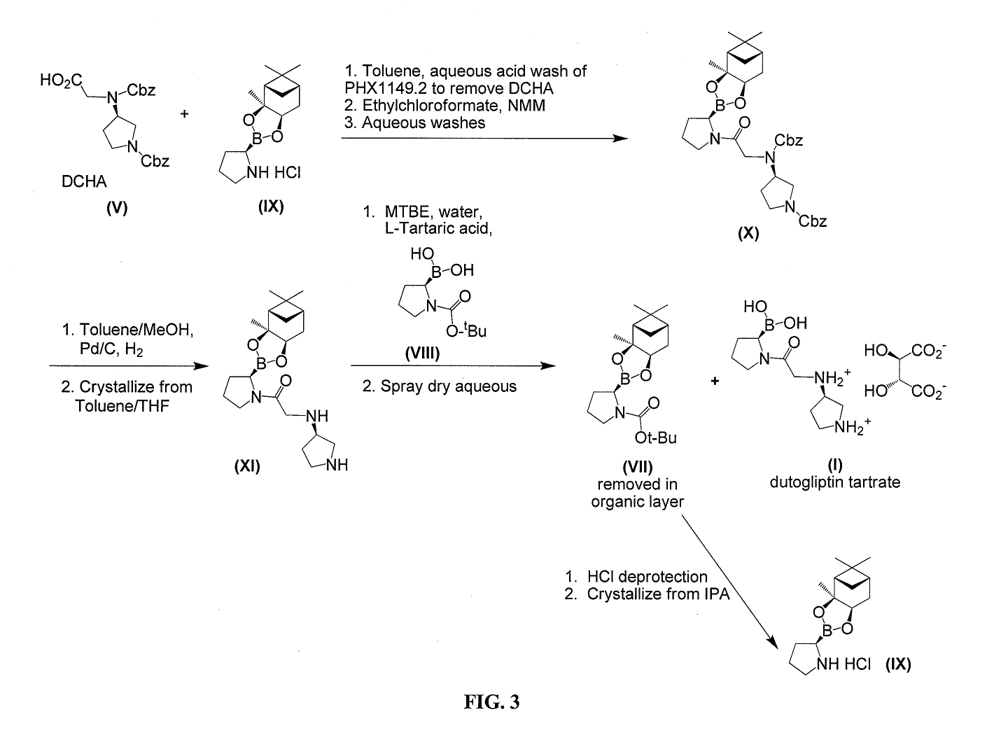

In some embodiments, as is depicted in FIG. 3, coupling of boronic ester (IX) with acid (V) proceeds under general amide coupling conditions to provide peptide (X). Removal of the CBz protecting groups yields compound (XI) which is subsequently converted to dutogliptin tartrate (I) by transesterification with boronic acid (VIII). The newly generated pyrrolidine (VII) can then be deprotected to generate pyrrolidine (IX) that can be recycled into the process.

### Preparation of the Acid of Formula (V)

In some embodiments, the acid of formula (V) used in reacting step (a) is prepared by:

(i) reacting the compound of formula (II) with any suitable source of R4 and/or R5 protecting groups, to form the compound of formula (III);

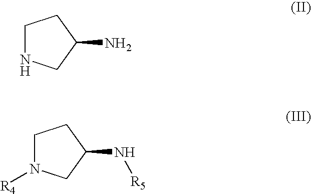

(ii) alkylating the compound of formula (III) to form the compound of formula (IV); and

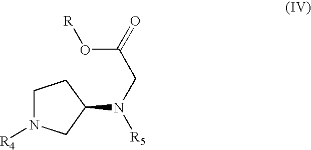

(iii) converting an ester of the compound of formula (IV) to a carboxylic acid or salt thereof, to form the compound of formula (V).

In some embodiments, reacting step (i) comprises protecting the aminopyrrolidone of formula (II) with any suitable source of a nitrogen protecting group. In some embodiments, the reacting step (i) comprises reacting the aminopyrrolidone of formula (II) with benzyl chloroformate, trifluoroacetic anhydride or t-butyl carbonate in the presence of a base.

In some embodiments, alkylating step (ii) comprises reacting the acylated pyrrolidine of formula (III) with an alpha-haloacetate (such as t-Butylbromoacetate, methylbromoacetate or chloroacetic acid).

In some embodiments, converting step (iii) comprises subjecting the aminoacetate of formula (IV) to acidic conditions. In particular, for example, converting step (iii) can comprise mixing the aminoacetate of formula (IV) with any suitable acid, for example, trifluoroacetic acid, hydrochloric acid or hydrobromic acid.

In some embodiments, the method comprises:

(i) reacting an aminopyrrolidine of formula (II) with benzyl chloroformate to form the compound of formula (III);

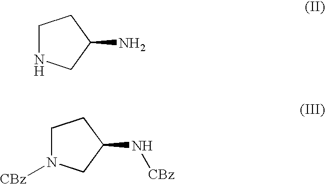

(ii) alkylating the compound of formula (III) to form the compound of formula (IV); and

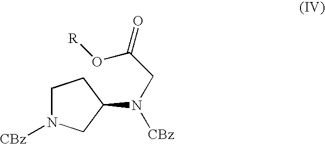

(iii) converting an ester of the compound of formula (IV) to a carboxylic acid, to form an acid of formula (V)

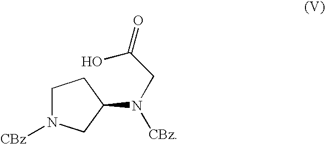

In some embodiments, the method comprises: (i) reacting an aminopyrrolidine of formula (II) with trifluoroacetic anhydride to form an acylated pyrrolidine of formula (III);

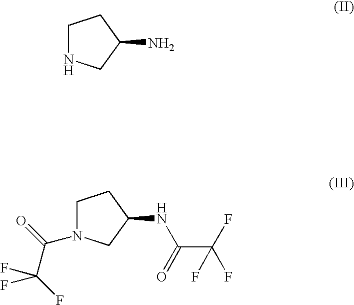

(ii) alkylating the acylated pyrrolidine of formula (III) to form an aminoacetate of formula (IV); and

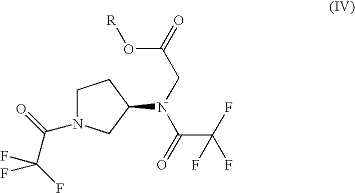

(iii) converting an ester of the aminoacetate of formula (IV) to a carboxylic acid, to form an acid of formula (V).

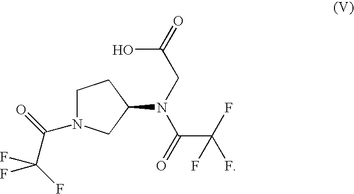

In some embodiments, the methods for preparing compound of formula (I), or a pharmaceutically acceptable salt, prodrug, solvate, and/or enantiomer thereof, comprises:

(a) reacting an aminopyrrolidine of formula (II) with benzyl chloroformate to form the compound of formula (III);

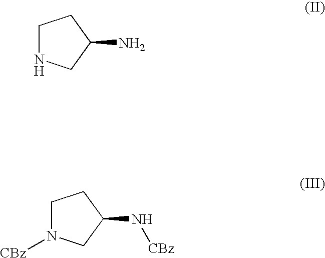

(b) alkylating the compound of formula (III) to form the compound of formula (IV);

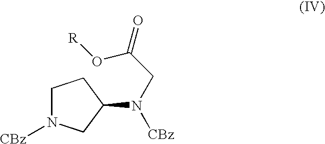

(c) converting an ester of the compound of formula (IV) to a carboxylic acid, to form the compound of formula (V);

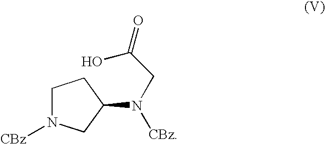

(d) converting the compound of formula (VI) to the compound of formula (VII) and/or the compound of formula (VIII);

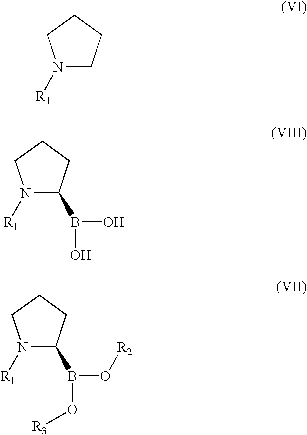

(e) optionally, if any compound of formula (VIII) is produced in step (d), converting the compound of formula (VIII) to the compound of formula (VII);

(f) deprotecting the R1 group of the compound of formula (VII) to form the boronic ester of formula (IX);

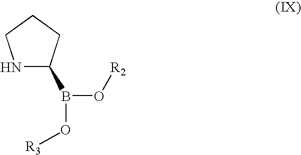

(g) reacting the boronic ester of formula (IX) and the acid of formula (V), to form the peptide of formula (X);

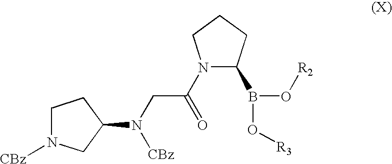

(h) removing the benzyl carbamate groups from the peptide of formula (X) to form the compound of formula (XI); and

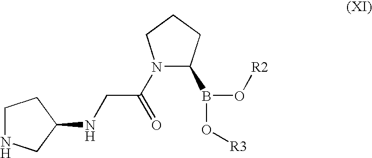

(i) converting the compound of (XI) to the compound represented by formula (I).

In some embodiments, the methods for preparing compound of formula (I), or a pharmaceutically acceptable salt, prodrug, solvate, and/or enantiomer thereof, comprises:

(a) reacting an aminopyrrolidine of formula (II) with trifluoroacetic anhydride to form an acylated pyrrolidine of formula (III);

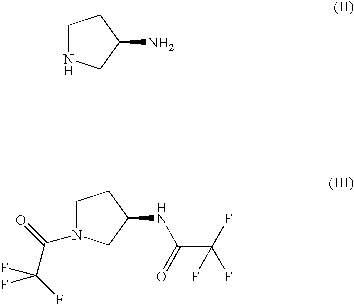

(b) alkylating the acylated pyrrolidine of formula (III) to form an aminoacetate of formula (IV);

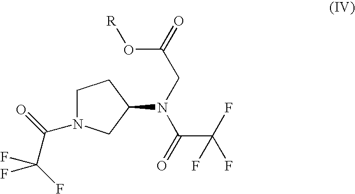

(c) converting an ester of the aminoacetate of formula (IV) to a carboxylic acid, to form an acid of formula (V);

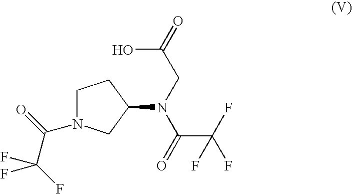

(d) reacting a pyrrolidine of formula (VI) with a chiral amine ligand and a base, and capturing the resulting anion with a boronic ester, to form a boronic ester of formula (VII) and/or a boronic acid of formula (VIII), wherein R1, R2, and R3 are protecting groups as discussed herein:

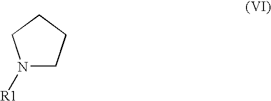

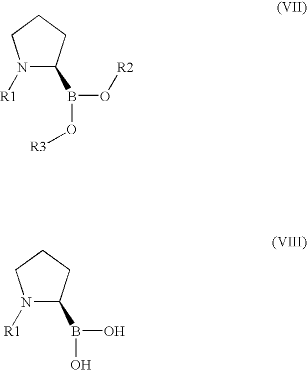

e) optionally, if any boronic acid of formula (VIII) is produced in step (d), converting the boronic acid of formula (VIII) to a boronic ester of formula (VII);
f) deprotecting the R1 group of the boronic ester of formula (VII) to form a boronic ester of formula (IX);

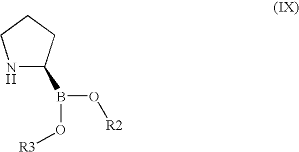

g) coupling the boronic ester of formula (IX) and the acid of formula (V), to form the peptide of formula (X);

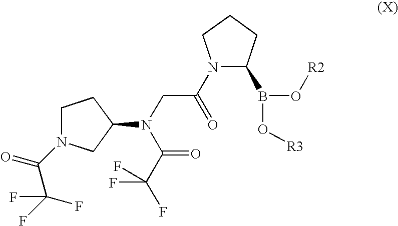

h) removing the trifluoroacetate groups from the peptide of formula (X) to form the boronic ester of formula (XI); and

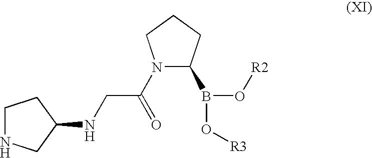

i) converting the boronic ester of (XI) to the boronic acid compound represented by formula (I).

In another embodiment, the present invention provides a method for preparing a pyrrolidine compound represented by formula (I)

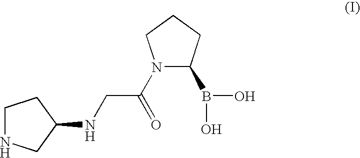

or a pharmaceutically acceptable salt, prodrug, solvate, and/or enantiomer thereof, wherein the method comprises: (a) reacting an acid of formula (V) with a boronic ester of formula (IX), to form a peptide of formula (X);

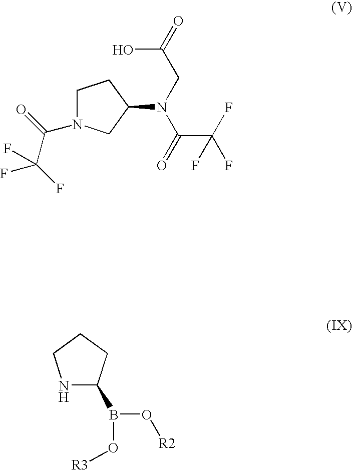

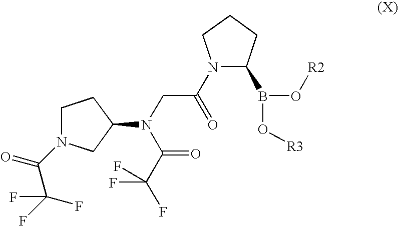

(b) removing the trifluoroacetate groups from the peptide of formula (X) to form the boronic ester of formula (XI); and

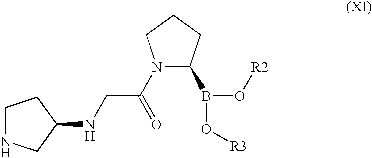

(c) converting the boronic ester of (XI) to the pyrrolidine compound represented by formula (I).

In some embodiments, the boronic ester of formula (IX) used and/or formed in the present invention is enantiomerically enriched.

In some preferred embodiments, the boronic ester of formula (IX) is formed by a method comprising:

(i) reacting a pyrrolidine of formula (VI) with a chiral amine ligand and a base, and capturing the resulting anion with a boronic ester, to form a boronic ester of formula (VII) and/or a boronic acid of formula (VIII), wherein R1, R2, and R3 are protecting groups as discussed herein;

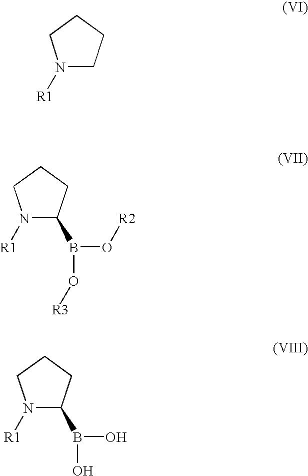

(ii) optionally, if any boronic acid of formula (VIII) is produced in step (i), converting the boronic acid of formula (VIII) to a boronic ester of formula (VII); and (iii) deprotecting the R1 group of the boronic ester of formula (VII) to form the boronic ester of formula (IX);

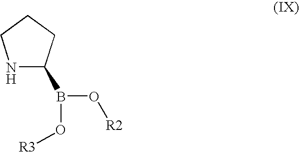

In some embodiments, the boronic acid of formula (VIII) or the boronic ester of formula (VII) formed by reacting step (i) is enantiomerically enriched.

In some embodiments, the acid of formula (V) is formed by a method comprising: (i) reacting an aminopyrrolidine of formula (II) with trifluoroacetic anhydride to form an acylated pyrrolidine of formula (III);

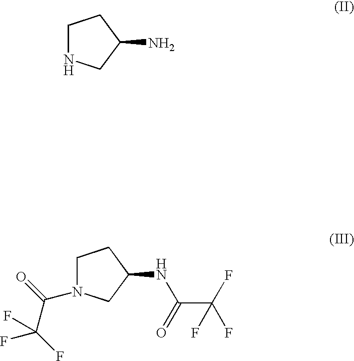

(ii) alkylating the acylated pyrrolidine of formula (III) to form an aminoacetate of formula (IV); and

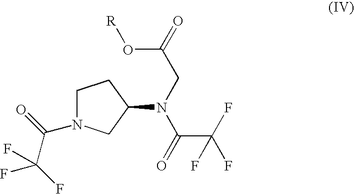

(iii) converting an ester of the aminoacetate of formula (IV) to a carboxylic acid, to form an acid of formula (V).

In another embodiment, the present invention provides a method for preparing a pyrrolidine compound represented by formula (I):

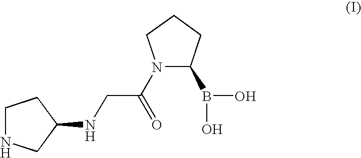

or a stereoisomer, pharmaceutically acceptable salt, prodrug, and/or solvate thereof, wherein the method comprises: reacting an aminopyrrolidine of formula (II) with trifluoroacetic anhydride to form an acylated pyrrolidine of formula (III).

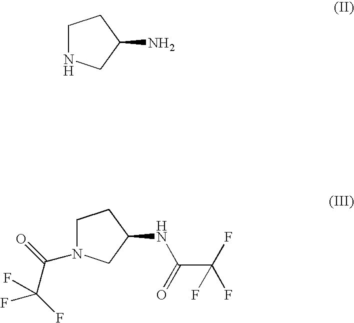

In another embodiment, the present invention provides a method for preparing a pyrrolidine compound represented by formula (I), comprising alkylating the acylated pyrrolidine of formula (III) to form an aminoacetate of formula (IV).

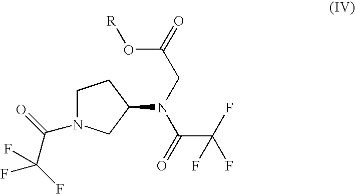

In another embodiment, the present invention provides a method for preparing a pyrrolidine compound represented by formula (I), comprising converting an ester of the aminoacetate of formula (IV) to a carboxylic acid, to form an acid of formula (V);

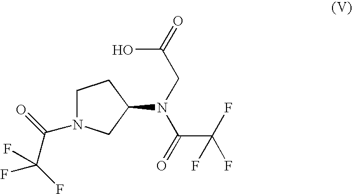

In another embodiment, the present invention provides a method for preparing a pyrrolidine compound represented by formula (I), comprising reacting a pyrrolidine of formula (VI) with a chiral amine ligand and a base, and capturing the resulting anion with a boronic ester, to form a boronic ester of formula (VII) and/or a boronic acid of formula (VIII), wherein R1, R2, and R3 are protecting groups as discussed herein;

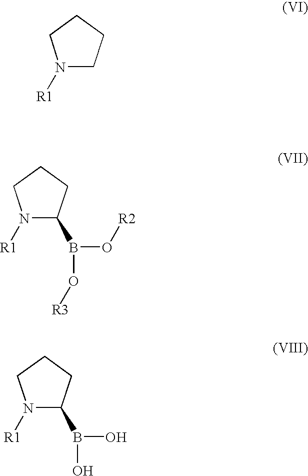

In another embodiment, the present invention provides a method for preparing a pyrrolidine compound represented by formula (I), comprising (a) coupling the boronic ester of formula (IX) and the acid of formula (V), to form the peptide of formula (X);

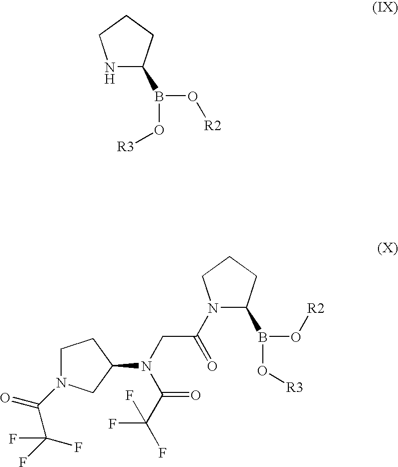

(b) removing the trifluoroacetate groups from the peptide of formula (X) to form the boronic ester of formula (XI); and

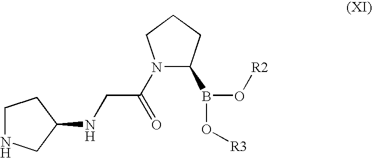

(c) converting the boronic ester of (XI) to the pyrrolidine compound represented by formula (I).

In another embodiment, the present invention provides a method for preparing a pyrrolidine compound represented by formula (I):

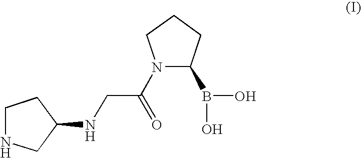

or a pharmaceutically acceptable salt, prodrug, solvate, and/or enantiomer thereof, wherein the method comprises: (a) reacting a pyrrolidine of formula (VI) with a chiral amine ligand and a base, and capturing the resulting anion, to form a boronic ester of formula (VII) and/or a boronic acid of formula (VIII), wherein R1, R2, and R3 are protecting groups as discussed herein;

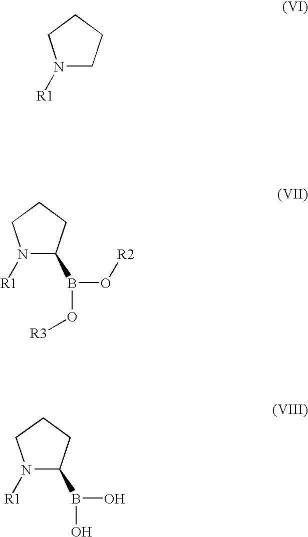

(b) optionally, if any boronic acid of formula (VIII) is produced in step (a), converting the boronic acid of formula (VIII) to a boronic ester of formula (VII);
(c) deprotecting the R1 group of the boronic ester of formula (VII) to form a boronic ester of formula (IX);

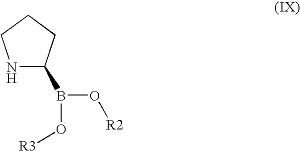

(d) coupling the boronic ester of formula (IX) and an acid of formula (V), to form the peptide of formula (X);

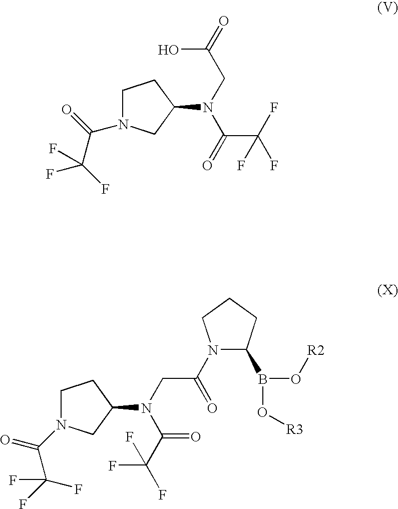

(e) removing the trifluoroacetate groups from the peptide of formula (X) to form the boronic ester of formula (XI); and

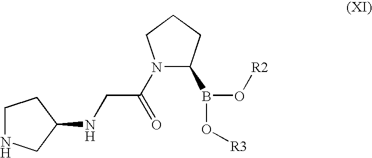

(f) converting the boronic ester of (XI) to the pyrrolidine compound represented by formula (I).

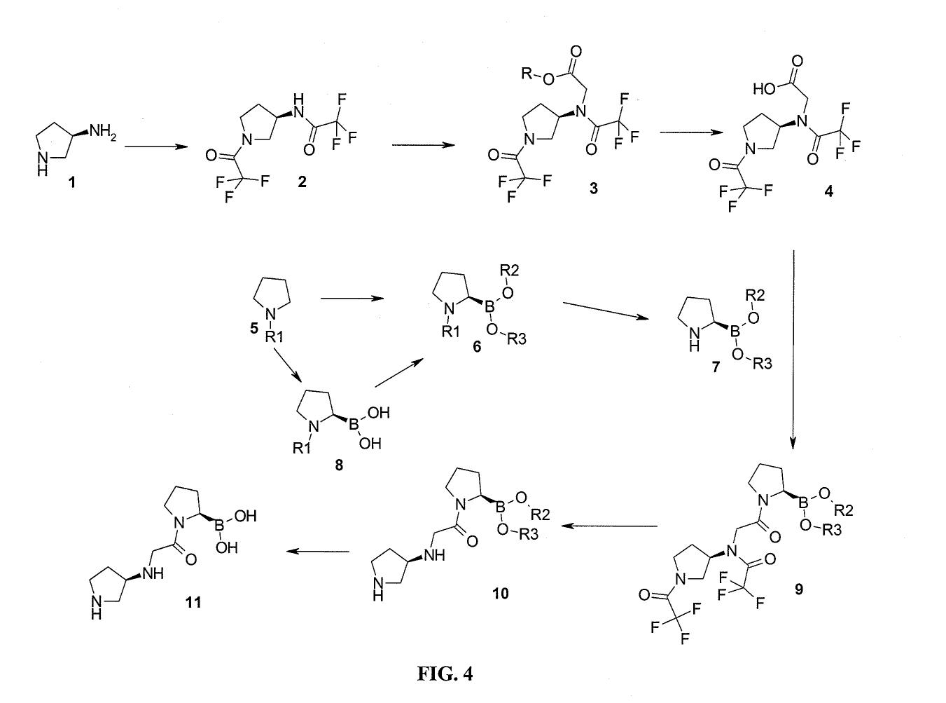

FIG. 4 provides a schematic of an example process for preparing the DPP-IV inhibitor of formula (I). As is illustrated, reaction of 3-R-aminopyrrolidine 1 with trifluoroacetic anhydride in the presence of a base (e.g., triethylamine, n-methylmorpholine or potassium carbonate) provides acylated pyrrolidine 2. Subsequent alkylation with an appropriate alpha-haloacetate (such as t-Butylbromo acetate or methylbromoacetate) yields the aminoacetate 3. Conversion of the ester to the carboxylic acid proceeds under acidic conditions (such as using trifluoroacetic acid, hydrochloric acid or hydrobromic acid) to provide acid 4. In a convergent manner pyrrolidine 5 is asymmetrically deprotonated with an appropriate chiral amine ligand (such as a chiral diamine ligand, e.g., (1S,2S)-Dimethyl-bis(3,3-dimethyl butyl)cyclohexane-1,2-diamine) and base (e.g., sec-butyl lithium) combination, and the resulting anion is captured with a boronic ester (such as trimethyl or triisopropyl borate) to form enantiomerically enriched boronic acid 8 or boronic ester 6. The boronic acid 8 is converted to boronic ester 6 with an appropriate diol. Deprotection of boronic ester 6, under acidic conditions, to form boronic ester 7. Coupling of boronic ester 7 with acid 4 proceeds under general amide coupling conditions (such as from mixed anhydrides, carbodiimides and acid chlorides) to provide peptide 9. Removal of the trifluoroacetate groups under basic conditions with ammonia, hydroxide or carbonate/alcohol yields boronic ester 10 which is subsequently converted to boronic acid 11 by reaction with phenyl boronic acid.

The present invention also provides compounds represented by formula (I) that are produced by the processes discussed herein, as well as pharmaceutical compositions and pharmaceutical formulations that comprise a pharmaceutically acceptable carrier and the compound of formula (I) produced by any of the processes discussed herein. Additionally, the present invention provides for the use of any of the intermediates discussed herein in the preparation and manufacturing of the compounds represented by formula (I).

The compound of formula (I) can be included in any suitable pharmaceutical composition or pharmaceutical formulation that include any desired other active components (e.g., medicaments or active agents) and/or inactive components (e.g., pharmaceutically acceptable carrier, excipients, diluents, binders, disintegrants, wetting agents, emulsifying agents, suspending agents, salts, buffering agents, coloring agents, sweetening agents, flavoring agents, or the like, etc.). In addition, the pharmaceutical composition or formulation can be in any desired form, for example, tablet, capsule, powder, sachet, aerosol, solution, suspension, paper, or topical composition, or container.

The pharmaceutical composition can comprise any desired inactive component(s). In some embodiments, the pharmaceutical composition comprises the compound of formula (I) and a pharmaceutical carrier. The pharmaceutical composition can be formulated with any one or more carriers such as conventional solid or liquid vehicles or diluents and pharmaceutical additives of a type appropriate to the mode of desired administration. Suitable carriers include, for example, any lactose, starch-based (e.g., corn starch or potato starch), talc and/or carbohydrate carrier, or any other carrier known to those of ordinary skill in the art. In some embodiments, the pharmaceutical composition comprises a medicinally inactive excipient, e.g., to dilute the API, assist in dispersion of the dosage form (e.g., tablet) in vivo (e.g., in the patient's stomach), bind the tablet together, and/or stabilize the API against degradation or decomposition. Any suitable diluent(s) can be included in the composition, e.g., diluents comprising microcrystalline cellulose (e.g., Avicel®), lactose, isomalt, and/or phosphate (e.g., monobasic calcium phosphate, dibasic calcium phosphate and tribasic calcium phosphate, or any orthophosphates, pyrophosphates, superphosphates, and/or polymeric phosphates, such as of calcium). Suitable binders for inclusion in the composition include, for example, copovidone. Any suitable disintegrants can be included in the compositions for example to facilitate dissolution of the dosage form after oral ingestion and/or to assist in hydration and to avoid the formation of gels in the stomach of the patient as the tablet dissolves, thus assisting in the release of the API into the gastric juices so that it can be absorbed into the bloodstream. Suitable disintegrants include, for example, crospovidone, cross-linked polyvinylpyrrolidine. Any suitable glidants can be used in the composition, for example, colloidal silicon dioxide or other fumed silica. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidine. Similarly, the carrier or diluent may include any sustained release material known in the art, such as glyceryl monostearate or glyceryl distearate, alone or mixed with a wax. In some embodiments, the composition comprises a tartrate or citrate salt of the compound of formula (I); a diluent (e.g., a binder comprising a microcrystalline cellulose); a binder (e.g., a binder comprising copovidone); a disintegrant (e.g., a disintegrant comprising crospovidone); a lubricant (e.g., a lubricant comprising magnesium stearate); and a glidant (e.g., a glidant comprising colloidal silicon dioxide). In some embodiments, the dosage form is free of calcium salts (e.g., calcium phosphate or calcium sulfate).

The pharmaceutical composition or formulation can comprise any desired additional medicaments and/or active agents, e.g., any active agent for treating, controlling, or preventing a disease, disorder, or condition that can be regulated or normalized via inhibition of DPP-IV. Suitable additional medicaments or active agents include, for example, any DPP-IV inhibitor other than Dutogliptin and/or any agent that increases insulin secretion and/or any anti-diabetic agent and/or any agent that reduces the uptake of sugar from the gastrointestinal track and/or any agent that enhances the effect of endogenous peptides or proteins that play a role in glycemic control and/or any agent that acts a replacement therapy for endogenous peptides or proteins that have a known role in glycemic control. Suitable agents include but are not limited to glyburide (e.g., Micronase® or Diabeta®), glipizide (e.g., Glucotrol®), nateglinide (e.g., Starlix®), repaglinide (e.g., Prandin®), metformin (e.g., Glucophage®), rosiglitazone (e.g., Avandia®), acarbose (e.g., Precose®), miglitol (e.g., Glyset®), exenatide (e.g., Byetta®), insulin (e.g., Humulin® or Novolin®), or combinations thereof. Suitable agents also include, for example, biguanides, chlorpropamide, glucagon-like peptide-1 (GLP-1) or mimetic thereof such as LY315902 or LY307161, glimepiride, meglitinide, phenformin, pioglitazone, sulfonyl urea, troglitazone, G1-262570, isaglitazone, JTT-501, NN-2344, L895645, YM-440, R-119702, AJ9677, KAD1129, APR-HO39242, GW-409544, KRP297, AC2993, Exendin-4, and NN2211. Such an additional medicament or active agent can be included in any therapeutically effective amount in the composition. In some embodiments, the pharmaceutical composition comprises the compound of formula (I) and a therapeutically effective amount of metformin. In some embodiments, the pharmaceutical composition comprises the compound of formula (I) and a therapeutically effective amount of pioglitazone. In some embodiments, the pharmaceutical composition comprises the compound of formula (I) and a therapeutically effective amount of a sulfonyl urea. The second medicament may be administered orally in the same dosage with the compound of formula (I), or in a separate oral dosage form. The compound of formula (I) and the second medicament may also be administered, for example by injection, separately, simultaneously or as a mixture.

In some embodiments, the composition comprises the compound of formula (I) and a therapeutically effective amount of an anti-obesity agent including but not limited to a beta 3 adrenergic agonist, a lipase inhibitor, a serotonin and/or dopamine reuptake inhibitors, a thyroid hormone receptor-beta agonist, an anorectic agent, a fatty acid oxidation up-regulator, or a mixture of any two or more thereof. Suitable anti-obesity agents include, for example, orlistat, sibutramine, topiramate, axokine, dexamphetamine, phentermine, phenylpropanolamine, famoxin, mazindol, or a mixture of any two or more thereof. These anti-obesity agents may be employed in the same dosage form with a compound of formula (I) or in different dosage forms.

In some embodiments, the composition comprises the compound of formula (I) and a therapeutically effective amount of an agent for treating polycystic ovary syndrome. Suitable agents for treating polycystic ovary syndrome include, for example, gonadotropin releasing hormones (GnRH), leuprolide (Lupron®), Clomid®, Parlodel®, oral contraceptives, or insulin sensitizers (e.g., PPAR agonists), or a combination or mixture thereof.

The composition can include any therapeutically effective amount of additional active agents. In some embodiments, the weight ratio of the compound of the formula (I) to the additional active agent within the composition is between about 0.01:1 and about 100:1, for example, between about 0.1:1 and about 5:1.

The use of a compound of formula (I) in combination with one or more other antidiabetic agents may produce antihyperglycemic results greater than that possible from each of these antidiabetic agents alone. The use of a compound of formula (I) in combination with one or more other antidiabetic agents may also produce a synergistic effect in that the antihyperglycemic result may be greater than the combined additive antihyperglycemic effects produced by these antidiabetic agents.

Pharmaceutical compositions containing a compound of formula (I) of the invention may be prepared by conventional techniques, as described, for example, in Remington: The Science and Practice of Pharmacy, 19th Ed., 1995. For example, tablets comprising the compound of formula (I) can be prepared by milling the tartrate salt of compound of formula (I) to provide a milled compound; blending the milled compound with a diluent (e.g., including microcrystalline cellulose) to provide a blended milled compound; granulating the blended milled compound in a fluidized bed granulator with a solution of binder (e.g., copovidone) in water to provide granules; then drying the granules; milling and screening the granules to provide dried, milled granules; blending the dried, milled granules with a dispersant (e.g., including crospovidone), glidant (e.g., including colloidal silicon dioxide), and lubricant (e.g., including magnesium stearate) to provide a lubricated blend; and then compressing the lubricated blend in a tablet press. In other embodiments, tablets comprising the compound of formula (I) can be prepared by dry mixing the compound of formula (I) (e.g., a tartrate salt of the compound of formula (I)), a diluent (e.g., including microcrystalline cellulose), and a binder (e.g., including copovidone) in a high shear granulator to provide a dry mix; adding water to the dry mix to provide granules; drying and milling the granules; adding a dispersant (e.g., including crospovidone), glidant (e.g., including colloidal silicon dioxide) and a lubricant (e.g., including magnesium stearate); mixing these components together to provide a lubricated blend; and compressing the lubricated blend in a tablet press. In other embodiments, tablets comprising the compound of formula (I) are prepared by dry granulating the compound of formula (I) and a diluent (e.g., including microcrystalline cellulose) using any suitable technique, e.g., roller compacting, to form dried granules; milling or grinding the dried granules into a powder; combining the powder with a dispersant, glidant, and lubricant as described herein; and compressing the lubricated blend into tablets. In some embodiments, the tablet is coated by any suitable coating agent, e.g., a polymer including but not limited to polyvinyl pyrrolidine, polyvinyl alcohol, hydroxypropyl methyl cellulose and/or hypromellose that can serve to preserve tablet integrity, reduce dusting, and repel moisture. Such coatings can be moisture-protective coatings.

The pharmaceutical composition or formulation can comprise any suitable concentration of the compound of formula (I) tartrate on a free base basis. In some embodiments, the composition comprises about 50-500 mg, for example, about 75-450 mg, about 100-400 mg, such as 50 mg, 100 mg, 200 mg, 400 mg, or 800 mg of the compound of formula (I) on a free base basis. A “free base” is the molecular form of an amine wherein the amine is not in salt form. When it is stated that an inventive dosage form contains some quantity of the compound of formula (I) tartrate “on a free base basis,” for example, what is meant is that the quantity of the tartrate salt form of the API that is included is equivalent to the stated quantity of the API in its free base form; i.e., that actual quantity of API tartrate in the dosage form is normalized for the difference in molecular weight between the free base and the tartrate salt of the free base of the compound of formula (I). Thus, for a monotartrate, non-hydrated form, the actual weight of the tartrate salt will be about 162% of the weight of the API on a free base basis, the ratio of the sum of the molecular weights of the compound of formula (I) and tartaric acid to the molecular weight of the compound of formula (I), i.e., about 390/240.

The composition can be in any desired form for delivery by any desired route of administration. In this regard, the route of administration may be any route, which effectively transports the compound of formula (I) to the appropriate or desired site of action, such as oral, nasal, pulmonary, buccal, rectal, subdermal, intradermal, transdermal or depot, subcutaneous, intravenous, intraurethral, intramuscular, intranasal, ophthalmic solution or an ointment, the oral route being preferred. In some embodiments, the composition can be in the form of a tablet, capsule, powder, sachet, aerosol, solution, suspension, paper, or topical composition, or container.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, a pharmaceutical composition may be a powder suitable for reconstitution with an appropriate solution as described above. Examples of these include, but are not limited to, freeze dried, rotary dried or spray dried powders, amorphous powders, granules, precipitates, or particulates. For injection, a pharmaceutical composition may optionally contain stabilizers, pH modifiers, surfactants, bioavailability modifiers and combinations of these. A compound of formula (I) may be formulated for parenteral administration by injection such as by bolus injection or continuous infusion. A unit dosage form for injection may be in ampoules or in multi-dose containers.

A pharmaceutical composition of the invention may include, for example, micelles or liposomes, or some other encapsulated form, or may be administered in an extended release form or an enteric coated form to provide a prolonged storage and/or delivery effect. Therefore, the pharmaceutical composition may be compressed into pellets or cylinders and implanted intramuscularly or subcutaneously as depot injections or as implants such as stents. Such implants may employ known inert materials such as silicones and biodegradable polymers, e.g., polylactide-polyglycolide. Examples of other biodegradable polymers include poly(orthoesters) and poly(anhydrides).

A compound of formula (I) may be formulated as a sustained release implant or implantable material suitable for continuous administration over a significant period of time. Typical sustained release implants are formed from polymers of pharmaceutically acceptable, biodegradable polymers such as polymers and copolymers of lactic acid, lactide, glycolic acid, glycolide, caproic acid and caprolactone. The dose and amount of compound of formula (I) within the implant will be calculated to deliver the desired single dose blood level of pyrrolidine compound.

For nasal administration, a pharmaceutical composition may contain a compound of formula (I) dissolved or suspended in a liquid carrier, in particular an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with a compound of formula (I) dissolved in polyhydroxylated castor oil.

The compound of formula (I) can be used to treat any diseases, disorders, or conditions (or symptom thereof) associated with DPP-IV and/or any diseases, disorders, or conditions (or symptom(s) thereof) that are amenable to treatment via inhibiting DPP-IV. For example, methods are provided for treating a mammal (e.g., a human) suffering from a disease, disorder, or condition that can be regulated or normalized via inhibition of DPP-IV such as any disease, disorder, or condition characterized by impaired glycemic control, for example diabetes mellitus and related conditions (e.g., Type 1 diabetes, Type 2 diabetes, gestational diabetes, Maturity Onset Diabetes of the Young (MODY), impaired glucose tolerance, impaired fasting glucose, hyperglycemia, impaired glucose metabolism, insulin resistance, obesity, diabetic complications, and the like) and/or diabetic complications and/or related conditions by administering a therapeutically effective amount of the compound of formula (I) to treat, control, ameliorate or prevent the disease, disorder, or condition. Such diseases, disorders, or conditions are known to be the result, at least in part, of the presence, or altered activity, of peptides regulated by the enzyme DPP-IV, for example in the context of its physiological role in glycemic control. In some embodiments, methods are provided for treating a disease, disorder, or condition in a mammal (e.g., human) by administering to the mammal (e.g., a human) a therapeutically effect amount of the compound of formula (I), e.g., a pharmaceutical composition comprising the compound of formula (I). Treatment is affected by inhibition of DPP-IV. Administration is typically accomplished through use of a pharmaceutical composition containing a compound of formula (I).

Methods are also included for selectively inhibiting DPP-IV over related enzymes through use of the compound of formula (I). In some embodiments, for example, DPP-IV is inhibited by greater than 5-fold relative to one or more other dipeptidyl peptidases. In other embodiments, DPP-IV is inhibited by greater than 10-, 20-, or even 50-fold or more over other dipeptidyl peptidases. Exemplary other dipeptidyl peptidases include DPP-VII, DPP-VIII, DPP-IX, and FAP. For example, a compound of formula (I) can selectively inhibit DPP-IV over dipeptidyl peptidase-VII, or DPP-IV over dipeptidyl peptidase-VIII, or DPP-IV over dipeptidyl peptidase-IX, or DPP-IV over fibroblast activation protein (FAP). In additional embodiments, a compound of formula (I) selectively inhibits DPP-IV over dipeptidyl peptidase-VIII and fibroblast activation protein. In other embodiments, the compound of formula (I) selectively inhibits DPP-IV over dipeptidyl peptidase-VII, dipeptidyl peptidase-VIII, and fibroblast activation protein. This selectivity applies to in vitro and to in vivo situations. In particular, it has been determined in an in vivo protocol study in humans that a compound of formula (I) maintained selectivity for inhibition of DPP-IV over the other amino dipeptidyl peptidases. Preferably, the DPP-IV selectivity is shown relative to DPP-VIII.

For in vivo use as a DPP-IV inhibitor, a compound of formula (I) may be formulated in any manner as described herein and administered in an effective amount to a patient (human) suffering from a disease, disorder, or condition that can be regulated or normalized by inhibition of DPP-IV, especially a disease, disorder, or condition characterized by impaired glycemic control, especially Diabetes Mellitus and related conditions. For example, the disease, disorder, or condition can be Type 1 diabetes, Type 2 diabetes, gestational diabetes, MODY, impaired glucose tolerance, impaired fasting glucose, hyperglycemia, impaired glucose metabolism, impaired glucose tolerance (IGT) and its progression to Type II diabetes, hyperinsulinemia, obesity, beta cell degeneration (in particular apoptosis of beta cells), the progression of non-insulin-requiring Type II diabetes to insulin requiring Type II diabetes; loss of the number and/or the size of beta cells in a mammalian subject, and diabetic complications such as retinopathy, neuropathy, nephropathy, cardiomyopathy, dermopathy, diabetes related infection, atherosclerosis, coronary artery disease, stroke and similar diseases, disorders, or conditions.

In other embodiments of method of treatment according to the invention, insulin resistance is a component of the disease, disorder, or condition that can be regulated or normalized by inhibition of DPP-IV. For example, the diseases, disorders, or conditions can be impaired fasting glucose, impaired glucose tolerance, polycystic ovarian syndrome and the like. In yet other embodiments, the disease, disorder, or condition that can be regulated or normalized by inhibition of DPP-IV involves a decrease of islet neogenesis, .beta.-cell survival, or insulin biosynthesis.

The administered dose of a compound of formula (I) will be carefully adjusted according to age, weight and condition of the patient, as well as the route of administration, dosage form and regimen and the desired result. The ultimate choice of dosage, route and pharmaceutical formulation will determined by the patient's attending physician, whose wisdom and judgment will guide this process. The dose for adults may range from about 0.5 to about 4,000 mg per day, for example about 0.5 to about 2,000 mg per day, preferably about 10 mg to about 1000 mg per day, more preferably about 50 mg to about 800 mg, for example, 50 mg, 100 mg, 200 mg, 400 mg, or 800 mg per day which can be administered in a single dose or in the form of multiple doses given up to 4 times per day. The compositions described above may be administered in the dosage forms as described above in single or divided doses of one to four times daily. It may be advisable, in some embodiments, to start a patient on a low dose combination and work up gradually to a high dose combination.

The administered dose of a compound of formula (I) within the pharmaceutical combination will be carefully adjusted according to age, weight and condition of the patient, as well as the route of administration, dosage form and regimen and the desired result. The ultimate choice of dosage, route and pharmaceutical formulation will determined by the patient's attending physician, whose wisdom and judgment will guide this process.

### EXAMPLES

The following examples are merely illustrative of aspects of the present invention and should not be construed as limiting the scope of the invention in any way as many variations and equivalents that are encompassed by the present invention will become apparent to those skilled in the art upon reading the present disclosure.

### Example 1

### Synthesis of (R)—N-(1,1-Dimethylethoxycarbonyl)(pyrrolidine-2-yl)boronic Acid

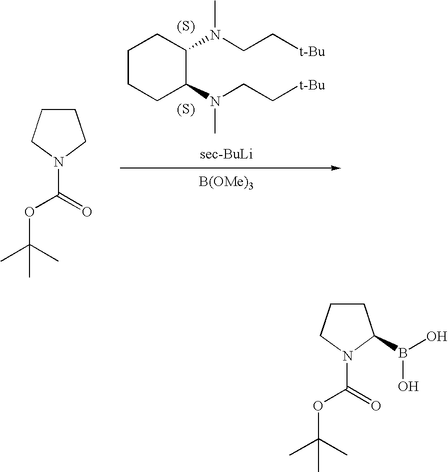

An oven dried 1 L three neck round bottom flask equipped with an overhead stirrer, addition funnel and internal thermocouple was charged with (1S,2S)-Dimethyl-bis(3,3-dimethylbutyl)cyclohexane-1,2-diamine (approx. 50 g, 161.23 mmol, 1.2 eq), BOC-pyrrolidine (approx. 23.55 ml, 134.35 mmol, 1 eq) and dry toluene (approx. 500 ml) under inert atmosphere. The clear colorless solution was cooled to 78° C. and a solution of sec-BuLi (approx. 115.16 ml of a 1.4 solution in cyclohexane, 161.23 mmol, 1.2 eq) was added slowly via dropping funnel over approx. 10 minutes (the temperature of the reaction mixture was maintained between approx. −78° C. and −65° C.). The light orange colored solution was stirred for 3.5 hours at approx. −78° C., which was then followed by the addition of a solution of trimethylborate (approx. 45.06 ml, 403.05 mmol, 3 eq) in toluene (approx. 75 ml) via dropping funnel over 30 minutes while maintaining the temperature below −65° C. The reaction mixture was warmed slowly to room temperature, and stirred for 16 hours at room temperature. The reaction mixture was added into an aqueous sodium hydroxide solution (approx. 670 ml of 2.0 M solution, 1340 mmol, 10 eq) and the resulting cloudy mixture was stirred for 30 minutes before allowing layers to separate. The aqueous phase (product) was transferred to a receiver and backwashed with toluene (approx. 100 ml). The organic phases (chiral amine ligand) were transferred to a receiver for later isolation. The aqueous phase was acidified to pH 5-6 by slow addition of HCl (conc.), then extracted with EtOAc (approx. 3×500 ml). The organic extracts were combined, dried over Na₂SO₄ and concentrated until a final volume of approximately 100 ml. Heptane (approx. 300 ml) was added and the concentrated mixture was stirred at room temperature overnight (approx. 15 hours). The resulting white precipitate was filtered and the filter cake was washed with cold heptane. The product was dried at room temperature under vacuum to yield (R)— (pyrrolidine-2-yl)boronic acid (approx. 20.31 g, 94.44 mmol, 70.27%) as a white solid. [α]²⁵D-72.5 (c 1, DCM); 94-95 ee (% ee was determined through chiral HPLC); ¹H NMR (400 MHz, D₂O) δ 3.40-3.50 (1H), 3.20-3.30 (1H), 2.90-3.00 (1H), 2.10 (1H), 2.00 (1H), 1.85 (1H), 1.72 (1H), 1.45-1.48 (9H); m/z (ES+) 216.06.

### Example 2

### Isolation of the chiral ligand ((1S,2S)-Dimethyl-bis(3,3-dimethyl butyl) cyclohexane-1,2-diamine)

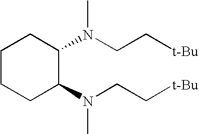

Water (approx. 300 ml) was added to the first organic extract from the previous workup and cooled to 0° C. the mixture was acidified to pH 3 by slow addition of HCl. The resulting cloudy mixture was stirred vigorously before allowing layers to separate. The aqueous phase (product) was transferred to a receiver and backwashed with toluene (approx. 100 ml). The aqueous phase was stirred at 0° C. and the pH of the solution was adjusted to 12-13 by the addition of sodium hydroxide. The mixture was extracted with toluene (approx. 3×500 ml) and the combined organic phases were concentrated under reduced pressure to give the crude chiral diamine (approx. 48.32 g, 155.57 mmol, 96.5%) as light yellow oil. Further purification by vacuum distillation (approx. 120-130° C., house vacuum) yielded the chiral diamine as a colorless oil (approx. 45.57 g, 146.72 mmol) in 91% recovery).

### Example 3

### Synthesis of (R)—N-(1,1-dimethylethoxycarbonyl)-pinanediol-(Pyrrolidin-2-yl) boronate

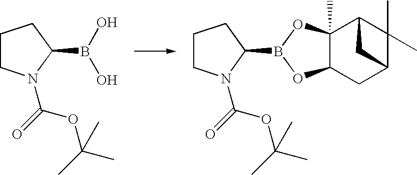

A solution of (R)-Pyrrolidine boronic acid (approx. 300 mg, 1.39 mmol) in isopropyl acetate (approx. 10 ml) was treated with (+)-pinanediol (approx. 236.35 mg, 1.39 mmol, 1 eq) and Na₂SO₄ (approx. 203.25 mg, 1.39 mmol, 1 eq). After 24 hr, the solvent was evaporated to give crude boronic ester (approx. 475.55 mg, 1.36 mmol, 98%) as a clear oil: 98-99% de via chiral HPLC; ¹H NMR (400 MHz, CDCl₃) δ 4.32 (1H), 3.47 (1H), 3.41-3.31 (2H), 3.22-3.05 (1H), 2.38-2.30 (1H), 2.20-1.75 (8H), 1.45 (9H), 1.41 (3H), 1.28 (3H), 0.85 (3H); m/z (ES, M+1) 350.28.

### Example 4

### (R)—N-(Pyrrolidine-2-yl)-pinacol boronate

To a solution of pyrrolidine boronic acid (approx. 456 mg, 2.12 mmol) in isopropyl acetate (approx. 15 ml) was added pinacol (approx. 251 mg, 2.12 mmol, 1 eq) and Na₂SO₄ (approx. 310 mg, 2.12 mmol, 1 eq). The mixture was stirred for 24 hr and the solvent was evaporated to yield crude pinacol boronate. The residue was triturated with EtOAc/hexane (approx. 1:10) at RT for 1 hr then filtered to give the pinacol boronate (approx. 611 mg, 2.06 mmol, 97%) as a white solid: ¹H NMR (400 MHz, CDCl₃) δ 3.40-2.95 (3H), 1.95-1.50 (4H), 1.40 (9H), 1.20 (12H); m/z (ES+) 298.21. Removal of the Boc-protecting group was achieved by dissolving the white solid pinacol boronate in dry ether (approx. 15 ml), cooling to 0° C. in an ice bath followed with addition of 1.5 eq of HCl in dioxane After 8 hours, the solvent was evaporated then triturated in hexane for 1 hr. The white precipitate was filtered and dried to yield the acid salt (approx. 472 mg, 2.02 mmol, 98%): ¹H NMR (CDCl₃) δ 3.48 (1H), 3.36 (1H), 3.21 (1H), 2.21 (1H), 2.03 (2H), 1.95 (1H), 1.35 (12H); m/z (ES M+1) 198.21.

### Example 5

### Synthesis of (R)-3-(Benzyloxycarbonyl-{2-oxo-2-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethyl}-amino)-pyrrolidine-1-carboxylic acid benzyl ester

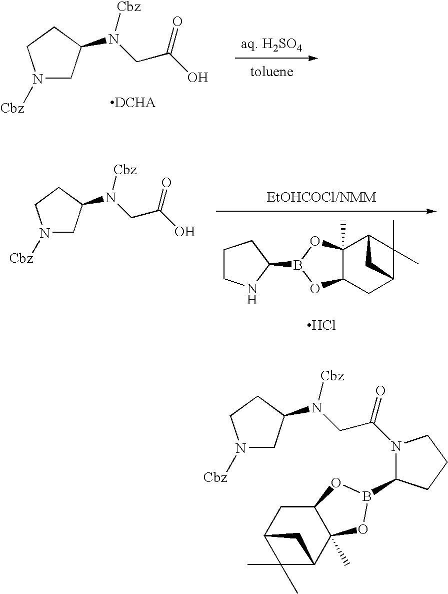

A mixture of (R)-3-(benzyloxycarbonyl-carboxymethyl-amino)-pyrrolidine-1-carboxylic acid benzyl ester dicyclohexylamine salt) (approx. 300.0 g, 0.505 mol), water (approx. 1.5 L), 2M aqueous sulfuric acid (approx. 0.75 L, 1.5 mol) and toluene (approx. 2 L) was stirred in a 10 L reactor at room temperature for 15 min. After settling the layers were separated. The aqueous layer was stirred with toluene (approx. 1.0 L) for 15 min, and the layers were separated. The combined organic layers were washed with water (approx. 1.5 L), and concentrated under vacuum at 45° C. to 1.5 L. To this solution was added N-methylmorpholine (approx. 55.4 mL, 0.505 mol) and this mixture was added to a cold solution (approx. 0°-5° C.) of ethyl chloroformate (approx. 48.1 mL, 0.505 mol) in toluene (approx. 1.0 L). The reaction mixture was stirred at 0°-5° C. for 15 min and solid (2-(2,9,9-trimethyl-3,5-dioxa-4-bora-tricyclo[6.1.1.0^{2,6}]dec-4-yl)-pyrrolidine hydrochloride) (approx. 144.4 g, 0.505 mol) was added in one portion followed by addition of N-Methylmorpholine (approx. 110.8 mL, 1.01 mol). The mixture was stirred for 30 min at 0°-5° C., and allowed to warm to 20°-25° C. Stirring was continued for an additional 2.5 h. Water (approx. 2.0 L) was then added, and the mixture was stirred for an additional 15 min. The layers were separated and the organic layer was subsequently washed with 0.85M aqueous sodium bicarbonate solution (approx. 1.2 L), water (approx. 2.0 L), and 0.065M citric acid solution (approx. 1.5 L). Toluene solution was concentrated under vacuum at 45° C., to give 287.3 g (approx. 88.4%) of the title compound. ¹H NMR (400 MHz, CDCl₃, ppm): mixture of rotomers, 7.35-7.25 (10H, m); 5.22-4.99 (4H, m); 4.60 (1H, d); 4.22 (1H, dd); 4.11-3.65 (3H, m); 3.60-3.00 (6H, m); 2.32-1.91 (8H, m); 1.89-1.67 (4H, m); 1.42-1.18 (6H, m); 0.84-0.72 (3H, m); m/z (M+H)=644.

### Example 6

### Synthesis of 2-((R)-Pyrrolidin-3-ylamino)-1-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethanone

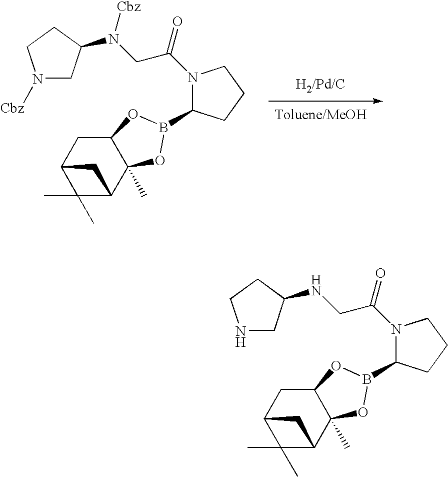

### a) THF Solvate

A solution of (R)-3-(Benzyloxycarbonyl-{2-oxo-2-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethyl}-amino)-pyrrolidine-1-carboxylic acid benzyl ester (approx. 4.76 g, 7.4 mmol) in toluene (approx. 60 mL) was diluted with methanol (approx. 60 mL). 10% Pd/C (wet, 500 mg) was added, and the mixture was hydrogenated at 50 psi for 3 h. The mixture was filtered through celite and washed with methanol (approx. 10 mL). The solution was then concentrated under vacuum to dryness. The residue was dissolved in THF (approx. 10 mL) at 40° C. and crystallized overnight at −10° C. to −15° C. Crystals were filtered, washed with cold THF (approx. 3 mL), and dried under vacuum for 5 h to yield 1.9 g (approx. 68.5%) of the title compound. ¹H NMR (400 MHz, D₂O, 1 drop TFA), δ 4.18-4.89 (m, 1H), 3.93-3.85 (m, 1H), 3.77 (s, 2H), 3.55 (dd, 1H), 3.45-3.38 (m, 4H), 3.35-3.25 (m, 2H), 3.24-3.05 (m, 3H), 2.93 (t, 1H), 2.33-2.24 (m, 1H), 2.15-1.42 (m, 16H), 1.09 (s, 3H), 0.94 (s, 3H), 0.78 (d, 1H), 0.50 (s, 3H). m/z (ES+)=376.30.

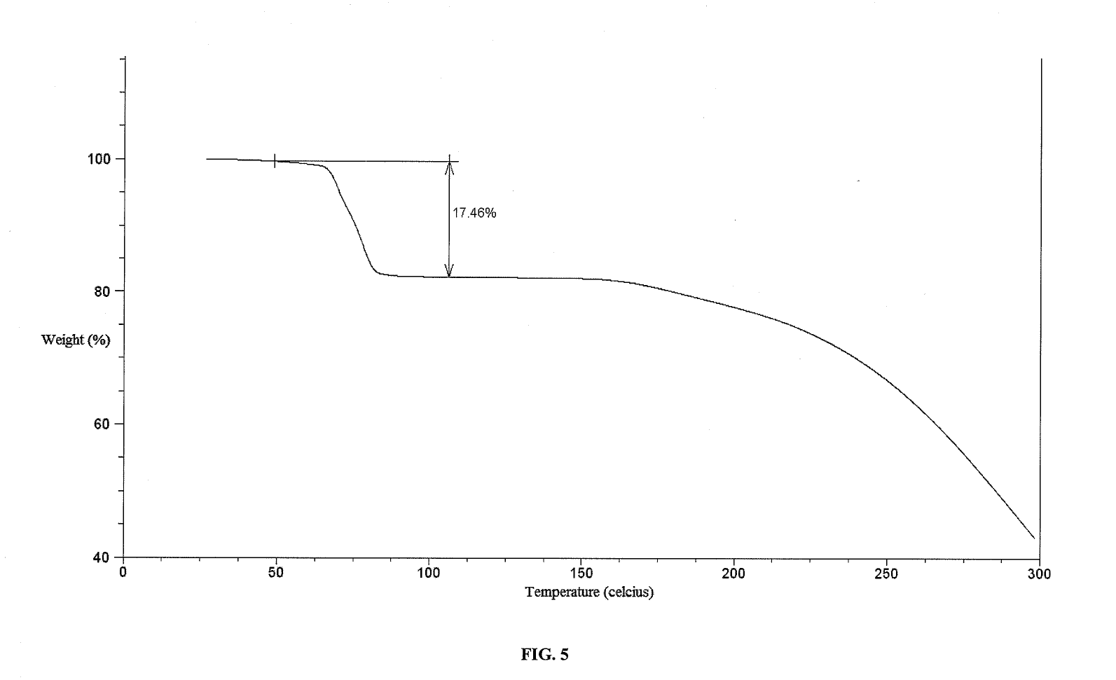

Thermogravimetric analysis of THF solvate of 2-((R)-Pyrrolidin-3-ylamino)-1-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethanone was performed as is shown in FIG. 5.

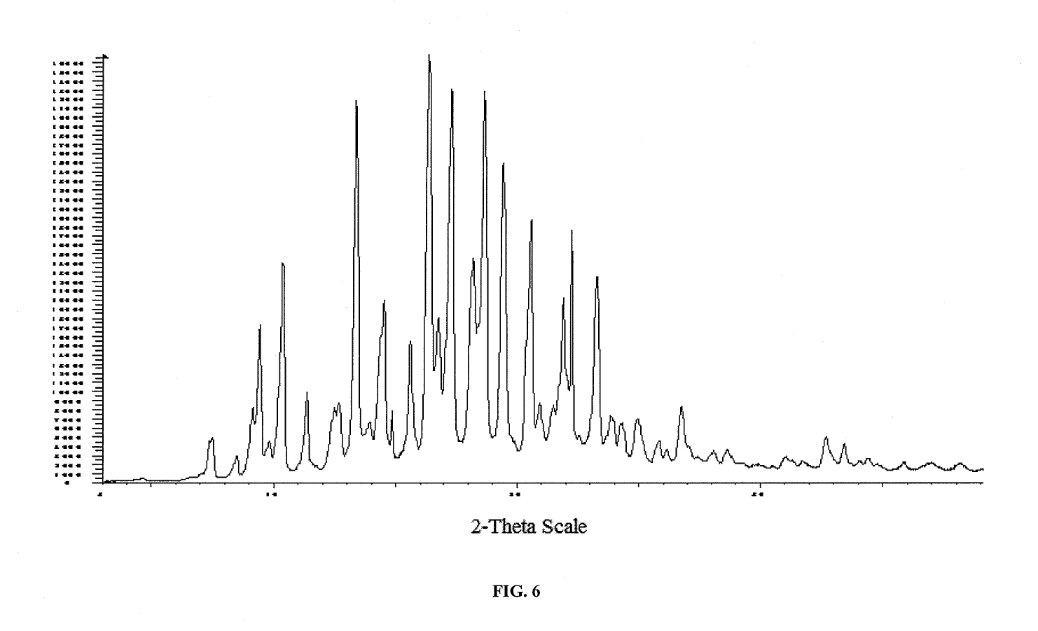

X-Ray Diffractogram of THF solvate of 2-((R)-Pyrrolidin-3-ylamino)-1-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethanone was performed as is shown in FIG. 6.

### b) Non-Solvate

A solution of (3-(Benzyloxycarbonyl-{2-oxo-2-[2-(2,9,9-trimethyl-3,5-dioxa-4-bora-tricyclo[6.1.1.0^{2,6}]dec-4-yl)-pyrrolidin-1-yl]-ethyl}-amino]-pyrrolidine-1-carboxylic acid benzyl ester) (approx. 20.0 g, 31.0 mmol) in toluene (approx. 80 mL) was diluted with methanol (approx. 20 mL). 10% Pd/C (2 g, wet) was added, and the mixture was hydrogenated at 50 psi for 3 h. The mixture was filtered through celite and the filter bed was washed with a mixture of toluene (approx. 20 mL) and methanol (approx. 4 mL). The solution was concentrated to 80 mL at 30-35° C. under vacuum (approx. 90 to 120 mBar). THF (approx. 100 mL) was added and the solution was concentrated to 120 mL at 30-35° C. under vacuum (approx. 90 to 120 mBar). The mixture was stirred at 35° C. for 1 h, resulting in crystallization. The mixture was cooled to 0° C. and held at that temperature for 2 h. Crystals were isolated by filtration, washed with a cold mixture of toluene (approx. 20 mL) and THF (approx. 5 mL), and dried under vacuum at 35° C. for 16 h to yield 9.11 g (approx. 24.3 mmol, 78%) of the title compound as a white solid. ¹H NMR (400 MHz, D₂O, 1 drop TFA), δ 4.34 (dd, 1H, J=9, 2 Hz), 4.08 (m, 1H), 3.99 (s, 2H), 3.74 (dd, 1H, J=13, 8 Hz), 3.52-3.29 (m, 6H), 3.12 (t, 1H, J=8 Hz), 2.47 (m, 1H), 2.27 (m, 1H), 2.19-2.06 (m, 2H), 2.02-1.84 (m, 6H), 1.67 (m, 2H), 1.30 (s, 3H), 1.15 (s, 3H), 1.00 (d, 1H, J=11 Hz), 0.71 (s, 3H). m/z (ES+)=376.30.

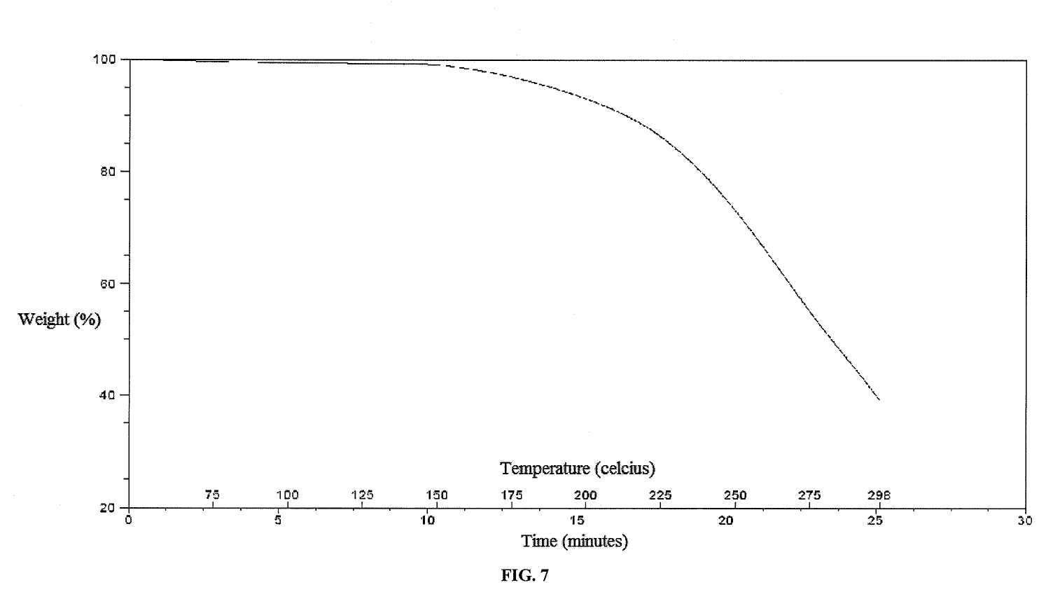

Thermogravimetric analysis of 2-((R)-Pyrrolidin-3-yl amino)-1-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethanone was performed as is shown in FIG. 7.

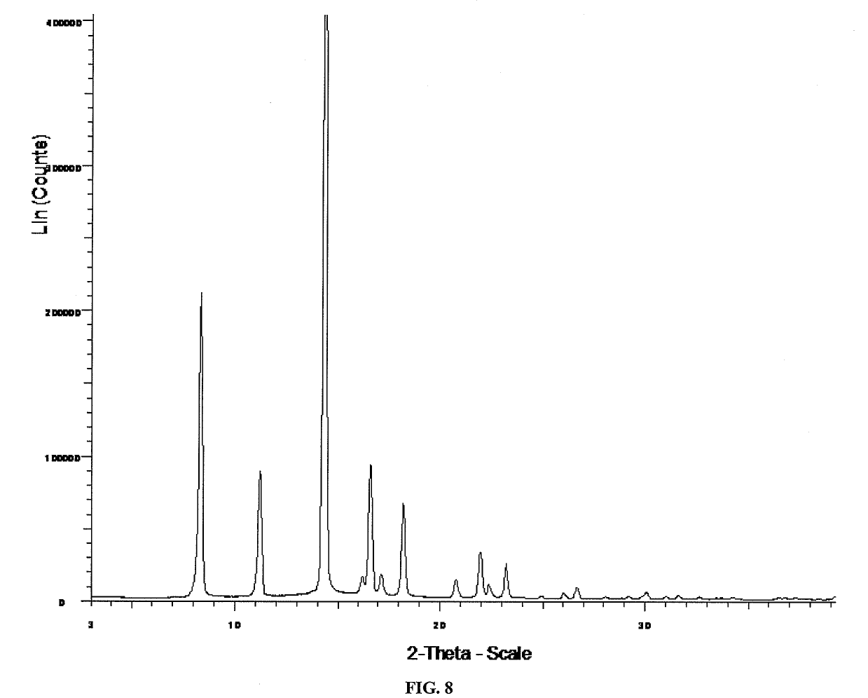

X-Ray Diffractogram of 2-((R)-Pyrrolidin-3-ylamino)-1-[(R)-2-((1S,2S,6R,8S)-2,9,9-trimethyl-3,5-dioxa-4-bora-tricyclo[6.1.1.0^{2,6}]dec-4-yl)-pyrrolidin-1-yl]-ethanone was performed as is shown in FIG. 8.

### Example 7

### Synthesis of Dutogliptin Tartrate

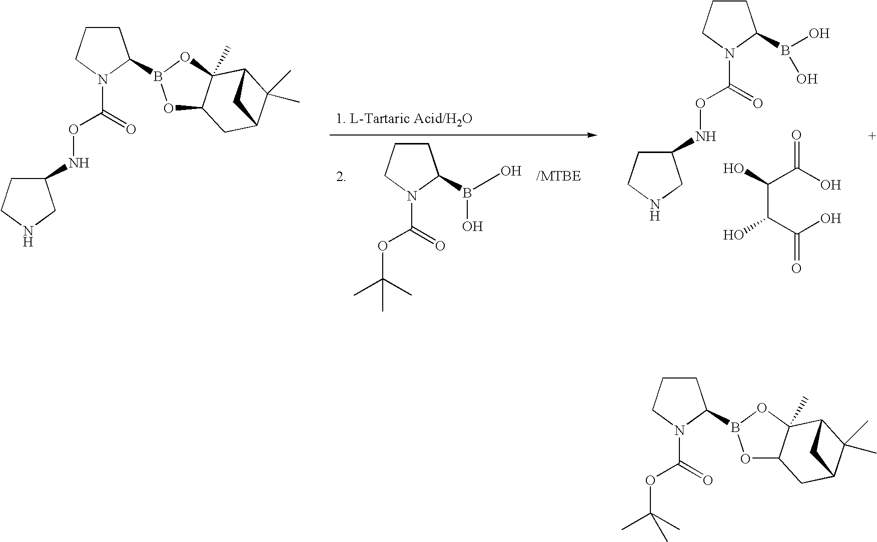

A round bottom flask equipped with a magnetic stirrer was charged with 2-(Pyrrolidin-3-ylamino)-1-[2-(2,9,9-trimethyl-3,5-dioxa-4-boratricyclo[6.1.1.0]dec-4-yl)-pyrrolidin-1-yl]-ethanone (approx. 1:1-Pinanediol borane/THF complex; 2.98 g, 6.67 mmol, 1 eq), (L)-tartaric acid (approx. 1.00 g, 6.67 mmol, 1 eq), and H₂O (approx. 15 mL). The mixture was allowed to stir for 1 hour then tert-Butyl methyl ether (approx. 15 ml) and (R)—N-(1,1-dimethylethoxycarbonyl)(pyrrolidine-2-yl)boronic acid (approx. 1.46 g, 6.80 mmol, 1.02 eq) were added. The bi-phasic mixture was allowed to stir for 20 hours at room temperature before separating the layers. The aqueous phase backwashed with tert-butyl methyl ether (approx. 15 ml) and the organic layers were combined. Lyophilization of the aqueous layer provided dutogliptin tartrate as a white solid (approx. 2.60 g, 6.65 mmol, 99.7%): ¹H NMR (400 MHz, D₂O, one drop of TFA) S 4.48 (2H), 3.95-3.88 (1H), 3.81 (2H), 3.59-3.54 (1H), 3.37-3.28 (2H), 3.21-3.16 (2H), 3.11-3.07 (1H), 2.82-2.78 (1H), 2.37-2.28 (1H), 2.04-1.96 (1H), 1.88-1.78 (2H), 1.71-1.60 (1H), 1.50-1.42 (1H); m/z (ES+) 241.10 (-tartrate acid).

### Example 8

### Synthesis of (R)—N-(Pyrrolidine-2-yl)-pinanediol boronate

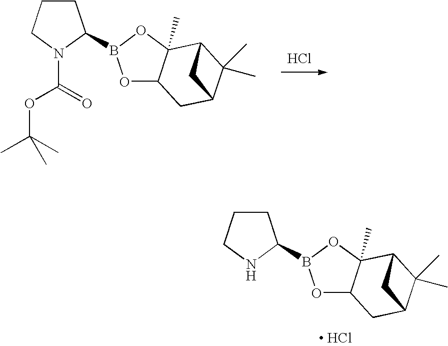

The organic layer (approx. 30 ml MTBE) from the previous organic extraction was cooled to 0° C. Ethanol (approx. 1.6 mL, 26.68 mmol, 4 eq) was added followed by drop-wise addition of acetyl chloride (approx. 1.89 mL, 26.68 mmol, 4 eq). The mixture was slowly warmed to room temperature and allowed to stir overnight (approx. 16 hrs). Isopropyl alcohol (approx. 30 ml) was added to the resulting slurry and stirred for 30 minutes and filtered. The filter cake was washed with cold isopropyl alcohol and the product was dried overnight under vacuum to give the title compound (approx. 1.75 g, 6.14 mmol, 92%) as a white solid. ¹H NMR (400 MHz, (CD₃)₂SO) δ 9.35 (1H), 8.57 (1H), 4.47-4.45 (1H), 3.16-3.01 (2H), 2.95-2.89 (1H), 2.35-2.29 (1H), 2.22-2.16 (1H), 2.09-2.03 (1H), 2.01-1.99 (1H), 1.89-1.67 (5H), 1.39 (3H), 1.26 (2H), 1.14-1.11 (1H), 0.83 (3H); m/z (ES+) 249.16.

### Example 9

### 2,2,2-Trifluoro-N—[(R)-1-(2,2,2-trifluoro-acetyl)-pyrrolidin-3-yl]-acetamide

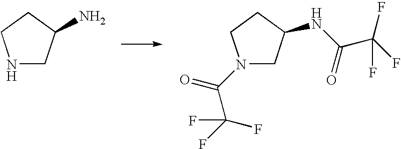

To a solution of 3-R-aminopyrrolidine (approx. 7.41 g, 86.2 mmol) and triethylamine (approx. 36 mL, 259 mmol) in DCM (approx. 100 mL) cooled in an ice bath was added a solution of trifluoroacetic anhydride (approx. 26 mL, 189.5 mmol) in DCM (approx. 20 mL) drop-wise over 30 min. The mixture was allowed to stir for 30 min then washed consecutively with water (approx. 50 mL), 2N HCl (approx. 50 mL) and sat. aq. sodium bicarbonate (approx. 50 mL). The organic layer was dried over anhydrous sodium sulfate and concentrated to yield the title compound (approx. 21.86 g, 91%). ¹H-NMR (400 MHz, DMSO-d6) (mixture of rotomers) δ: 9.73-9.69 (m, 1H), 4.50-4.35 (m, 1H), 3.90-3.86 (m, 0.5H), 3.74-3.69 (m, 1.5H), 3.63-3.45 (m, 2H), 2.30-2.11 (m, 1H), 2.11-1.90 (m, 1H); m/z (M+1)=279.02.

### Example 10

### {(2,2,2-Trifluoro-acetyl)-[(R)-1-(2,2,2-trifluoro-acetyl)-pyrrolidin-3-yl]-amino}-acetic acid tert-butyl ester

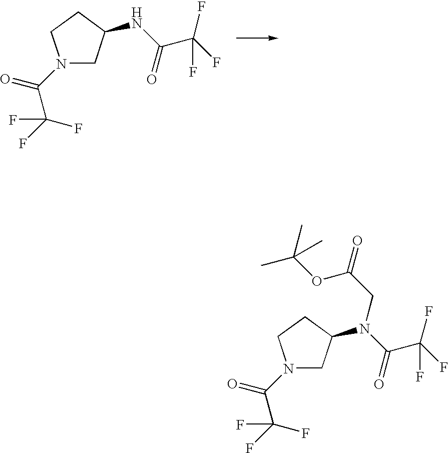

To a solution of acetate 2,2,2-trifluoro-N—[(R)-1-(2,2,2-trifluoro-acetyl)-pyrrolidin-3-yl]-acetamide (approx. 21.8 g, 78.6 mmol) in DMF (approx. 150 mL) was added a solution of t-butylbromoacetate (approx. 15.1 mL, 102 mmol) drop-wise over 1 h. The mixture was allowed to stir at ambient temperature for 15 h and filtered through a pad of celite. The filtrate was diluted with EtOAc (approx. 100 mL) and washed with water (approx. 100 mL) and brine (approx. 100 mL). The organic layer was dried over anhydrous sodium sulfate and concentrated. The residue was re-crystallized from isopropylether to yield the title compound (approx. 22.7 g, 73.7%) as a white solid. ¹H-NMR (400 MHz, DMSO-d6) (mixture of rotomers) δ: 4.90-4.68 (m, 1H), 4.33-4.27 (m, 0.7H), 4.14-4.12 (m, 1.3H), 3.90-3.55 (m, 3H), 3.49-3.40 (m, 1H), 2.26-2.04 (m, 2H), 1.42-1.39 (m, 9H); m/z (M−1)=391.08.

### Example 11

### {(2,2,2-Trifluoro-acetyl)-[(R)-1-(2,2,2-trifluoro-acetyl)-pyrrolidin-3-yl]-amino}-acetic acid

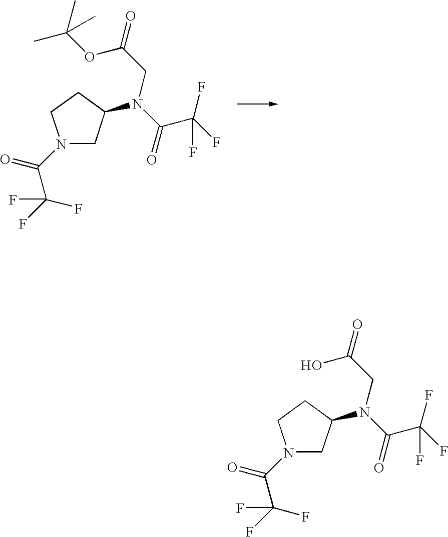

To a solution of {(2,2,2-trifluoro-acetyl)-[(R)-1-(2,2,2-trifluoro-acetyl)-pyrrolidin-3-yl]-amino}-acetic acid (approx. 12.37 g, 31.56 mmol) in toluene (approx. 100 mL) was added trifluoroacetic acid (approx. 20 mL). The mixture was allowed to stir for 15 h and concentrated to yield the title compound (approx. 10.5 g, 99%) as an off-white amorphous solid. ¹H-NMR (400 MHz, DMSO-d6) (mixture of rotomers) δ: 13.5-12.75 (m, 1H), 4.83-4.59 (m, 1H), 4.30 (s, 0.7H), 4.15 (s, 1.3H), 3.9-3.39 (m, 4H), 2.35-2.05 (m, 2H); m/z (M+1)=337.00.

### Example 12

### Synthesis of 2,2,2-Trifluoro-N-{2-oxo-2-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethyl}-N—[(R)-1-(2,2,2-trifluoro-acetyl)-pyrrolidin-3-yl]-acetamide

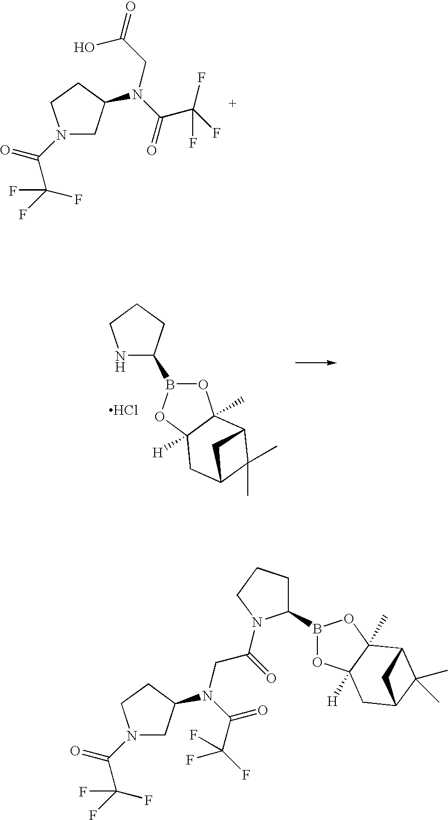

### Procedure A

To a solution of {(2,2,2-Trifluoro-acetyl)-[(R)-1-(2,2,2-trifluoro-acetyl)-pyrrolidin-3-yl]-amino}-acetic acid (approx. 2.48 g, 7.39 mmol) in DCM (approx. 50 mL) was added EDC (approx. 4.26 g, 22.17 mmol), HOBt (approx. 1.50 g, 11.09 mmol) (R)-2-((1S,2S,8S)-2,9,9-Trimethyl-3,5-dioxa-4-bora-tricyclo[6.1.1.0^{2,6}]dec-4-yl)-pyrrolidine hydrochloride (approx. 2.11 g, 7.39 mmol) and N-methylmorpholine (approx. 6.2 mL, 44.3 mmol). The mixture was stirred at 0° C. for 1 h and washed consecutively with sat. aq. sodium bicarbonate (approx. 50 mL), water (approx. 50 mL) and sat. aq. citric acid (approx. 50 mL). The organic layer was dried over anhydrous sodium sulfate and concentrated to yield the title compound (approx. 4.03 g, 96%) as a white solid. ¹H-NMR (400 MHz, DMSO-d6) (mixture of rotomers) δ: 4.80-4.62 (m, 1H), 4.40-4.15 (m, 3H), 3.97-3.21 (m, 7H), 3.01-2.90 (m, 1H), 2.33-1.58 (m, 12H), 1.35-1.18 (m, 8H), 0.82-0.78 (m, 3H); m/z (M+1)=568.06.

### Procedure B

A solution of {(2,2,2-Trifluoro-acetyl)-[(R)-1-(2,2,2-trifluoro-acetyl)-pyrrolidin-3-yl]amino}-acetic acid (approx. 4.65 g, 13.84 mmol) in DCM (approx. 40 mL) was added (chloromethylene)dimethyl-ammonium chloride (approx. 2.21 g, 17.30 mmol). The mixture was allowed to stir at ambient temperature for 15 min. After this time the solution was cooled in an ice bath and (R)-2-((1S,2S,8S)-2,9,9-Trimethyl-3,5-dioxa-4-bora-tricyclo[6.1.1.0^{2,6}]dec-4-yl)-pyrrolidine hydrochloride (approx. 3.94 g, 13.84 mmol) was added followed by triethylamine (approx. 5.80 mL, 41.5 mmol). The mixture was allowed to stir for 1 h then washed with water (approx. 100 mL), sat. aq. citric acid (approx. 100 mL) and sat. aq. sodium bicarbonate (approx. 100 mL). The organic layer was dried over anhydrous sodium sulfate and concentrated to yield the title compound (approx. 6.33 g, 81%) as a light yellow solid.

### Example 13

### Synthesis of 2-((R)-Pyrrolidin-3-ylamino)-1-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethanone

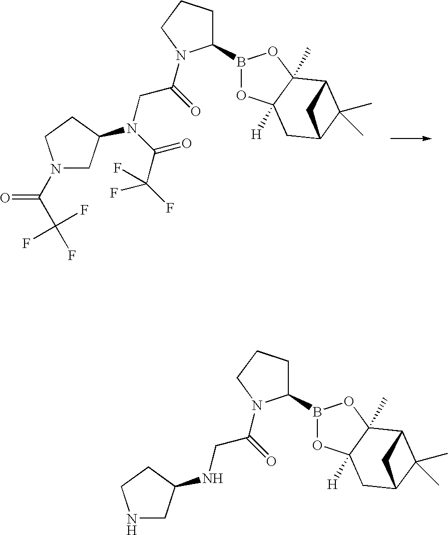

### Procedure A

To a solution of 2,2,2-Trifluoro-N-{2-oxo-2-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethyl}-N—[(R)-1-(2,2,2-trifluoro-acetyl)-pyrrolidin-3-yl]-acetamide (approx. 1.0 g, 1.76 mmol) in MeOH (approx. 20 mL) and water (approx. 2 mL) was added potassium carbonate (approx. 1.22 g, 8.82 mmol). The mixture was stirred at ambient temperature for 24 h. After this time the mixture was filtered and concentrated. The residue was then triturated with dichloromethane (approx. 30 mL) and filtered. The filtrate was concentrated to yield the title compound (approx. 502 mg, 76%) as an off-white solid. ¹H NMR (400 MHz, D₂O, 1 drop TFA), δ 4.34 (dd, 1H, J=9, 2 Hz), 4.08 (m, 1H), 3.99 (s, 2H), 3.74 (dd, 1H, J=13, 8 Hz), 3.52-3.29 (m, 6H), 3.12 (t, 1H, J=8 Hz), 2.47 (m, 1H), 2.27 (m, 1H), 2.19-2.06 (m, 2H), 2.02-1.84 (m, 6H), 1.67 (m, 2H), 1.30 (s, 3H), 1.15 (s, 3H), 1.00 (d, 1H, J=11 Hz), 0.71 (s, 3H). m/z (ES+)=376.30.

### Procedure B

A solution of To a solution of 2,2,2-Trifluoro-N-{2-oxo-2-[(R)-2-((1S,2S,8S)-2,9,9-trimethyl-3,5-dioxa-4-bora-tricyclo[6.1.1.0^{2,6}]dec-4-yl)-pyrrolidin-1-yl]-ethyl}-N—[(R)-1-(2,2,2-trifluoro-acetyl)-pyrrolidin-3-yl]-acetamide (approx. 200 mg, 0.353 mmol) in 2.0M ammonia solution in methanol (approx. 10 mL) was stirred at ambient temperature for 20 h. After this time the solution was concentrated to yield the title compound.

The entire disclosures of all applications, patents and publications, cited above and below, are hereby incorporated by reference.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A method for preparing the compound of formula (I): 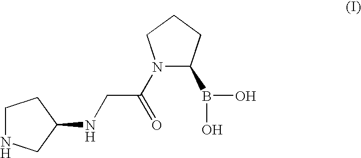
or a pharmaceutically acceptable salt thereof, wherein the method comprises:
(a) coupling the compound of formula (IX) with the compound of formula (V) to form the compound of formula (X): 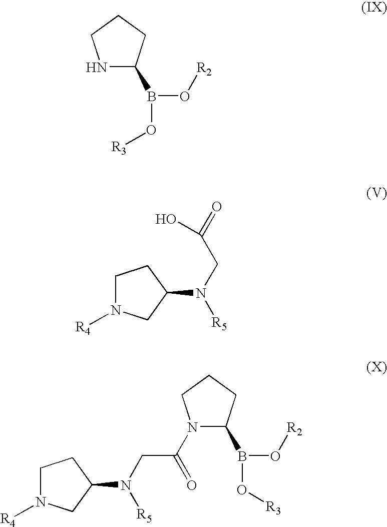
wherein R2, R3, R4 and R5 are protecting groups,
(b) removing the R4 and R5 groups from the compound of formula (X) to form the compound of formula (XI); 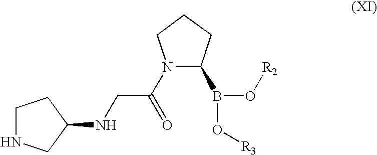
(c) reacting the compound of (XI) with an acid to form the compound of formula (I) and optionally the compound of formula (VII); 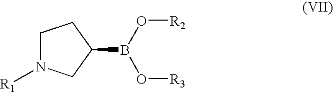
wherein R1 is a protecting group;
(d) optionally, if any compound of formula (VII) is formed in reacting step (c), removing the R1 group from the compound of formula (VII) to form the compound of formula (IX); and
(e) optionally recycling the compound of formula (IX) for use in reacting step (a).

2. The method of claim 1, wherein the compound of formula (VII) is formed in reacting step (c), and wherein removing step (d) and recycling step (e) are performed.

3. The method of claim 1, wherein the acid used in reacting step (c) is a boronic acid.

4. The method of claim 3, wherein the boronic acid is the compound of formula (VIII):

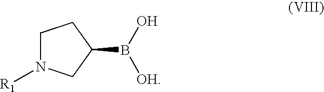

5. The method of claim 4, wherein the compound of formula (VIII) is in enantiomerically enriched form.

6. The method of claim 1, wherein the compound of formula (VII) formed in reacting step (c) is in enantiomerically enriched form.

7. The method of claim 1, wherein reacting step (a) comprises reacting the compound of formula (IX) and the compound of formula (V) under amide coupling conditions.

8. The method of claim 1, wherein reacting step (a) comprises coupling the compound of formula (IX) and the compound of formula (V) using an anhydride, a carbodiimide and/or an acid halide.

9. The method of claim 1, wherein the method further comprises preparing the compound of formula (IX) used in reacting step (a) by asymmetric synthesis.

10. The method of claim 1, wherein the method further comprises preparing the compound of formula (IX) used in reacting step (a) by a method comprising asymmetrically deprotonating the compound of formula (VI)

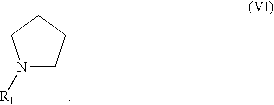

11. The method of claim 1, wherein the compound of formula (IX) used in reacting step (a) is prepared by:
(i) converting the compound of formula (VI) to the compound of formula (VII) and/or the compound of formula (VIII): 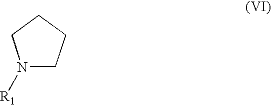 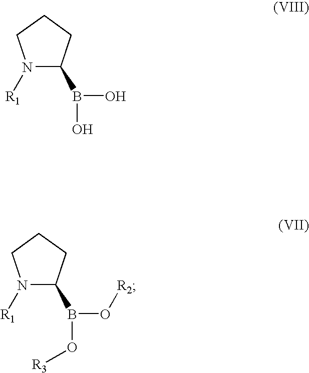
(ii) optionally, if any compound of formula (VIII) is produced in step (i), converting the compound of formula (VIII) to the compound of formula (VII); and
(iii) deprotecting the compound of formula (VII) to form the boronic ester of formula (IX).

12. The method of claim 11, wherein the compound of formula (VII) is in enantiomerically enriched form.

13. The method of claim 11, wherein the converting step comprises asymmetrically deprotonating the compound of formula (VI) and optionally capturing the resulting anion with a borate compound.

14. The method of claim 11, wherein the converting step comprises asymmetrically deprotonating the compound of formula (VI) with a chiral ligand and a base and optionally capturing the resulting anion with a borate compound.

15. The method of claim 11, wherein the compound of formula (IX) is in enantiomerically enriched form.

16. A method for preparing a compound represented by formula (I): 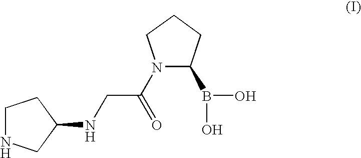
or a pharmaceutically acceptable salt thereof, wherein the method comprises:
(i) reacting the compound of (XI) with an acid to form the compound of formula (I) and optionally the compound of formula (VII):

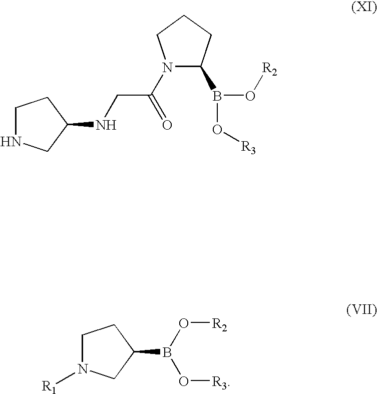

17. The method of claim 16, wherein the acid used in reacting step (i) is the boronic acid of formula (VIII):

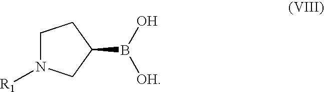

18. The method of claim 17, wherein the compound of formula (VIII) is in enantiomerically enriched form.

19. The method of any of claim 16, wherein the compound of formula (VII) is formed in reacting step (i).

20. The method of claim 19, wherein the compound of formula (VII) formed in reacting step (i) is in enantiomerically enriched form.

21. The method of claim 16, wherein the compound of formula (I) and the compound of formula (VII) formed in reacting step (i) are in enantiomerically enriched form.

22. The method of claim 16, wherein the compound of (XI) is prepared by
(a) reacting the compound of formula (IX) with the compound of formula (V), to form the compound of formula (X): 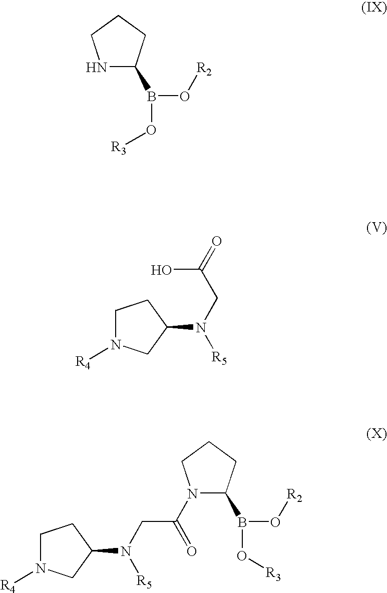
wherein R2, R3, R4 and R5 are protecting groups; and
(b) removing the R4 and R5 groups from the compound of formula (X) to form the compound of formula (XI).

23. The method of claim 22, wherein the compound of formula (IX) used in the reacting step is prepared by asymmetrically deprotonating the compound of formula (VI) and optionally capturing the resulting anion with a borate compound

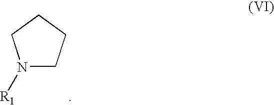

24. The method of claim 22, wherein the compound of formula (IX) used in the reacting step is prepared by asymmetrically deprotonating the compound of formula (VI) with a chiral ligand and a base and optionally capturing the resulting anion with a borate compound.

25. The method of claim 22, wherein the compound of formula (VII) is formed in reacting step (i), and wherein the method further comprises:
removing the R1 group from the compound of formula (VII) to form the compound of formula (IX); and
recycling the compound of formula (IX) for use in reacting step (a).

26. The method of claim 22, wherein reacting step (a) comprises reacting the compound of formula (IX) and the compound of formula (V) under amide coupling conditions.

27. The method of claim 22, wherein reacting step (a) comprises coupling the compound of formula (IX) and the compound of formula (V) using an anhydride, a carbodiimide, and/or an acid halide.

28. The method of claim 22, wherein the boronic ester of formula (IX) used in reacting step (a) is prepared by:
(1) converting the compound of formula (VI) to the compound of formula (VII) and/or the compound of formula (VIII); 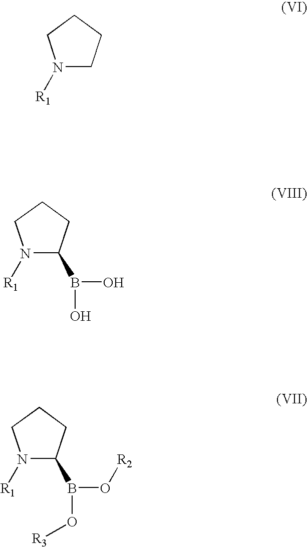
(2) optionally, if any compound of formula (VIII) is produced in step (i), converting the compound of formula (VIII) to the compound of formula (VII); and
(3) deprotecting the R1 group of the compound of formula (VII) to form the boronic ester of formula (IX).

29. The method of claim 28, wherein the compound of formula (VII) formed in steps (1)-(2) is in enantiomerically enriched form.

30. The method of claim 28, wherein the compound of formula (IX) formed in step (3) is in enantiomerically enriched form.

31. A pyrrolidine compound represented by formula (I) that is produced by the process of claim 1.

32. A pharmaceutical composition comprising a pharmaceutically acceptable carrier and a compound of formula (I) that is produced by the process of claim 1.

33. A method for preparing a compound represented by formula (I): 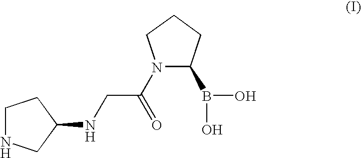
or a pharmaceutically acceptable salt thereof, wherein the method comprises:
(i) converting the compound of formula (VI) to the compound of formula (VII) and/or the compound of formula (VIII);

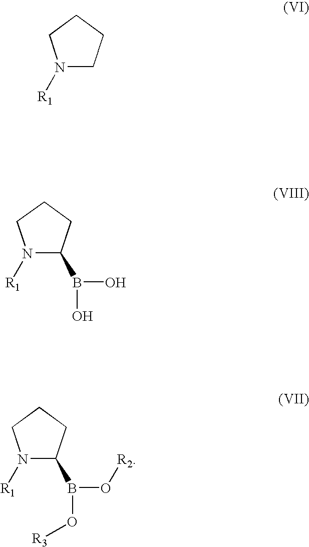

34. The method of claim 33, wherein the converting step comprises asymmetrically deprotonating the compound of formula (VI) and optionally capturing the resulting anion with a borate compound.

35. The method of claim 33, wherein the converting step comprises asymmetrically deprotonating the compound of formula (VI) with a chiral ligand and a base and optionally capturing the resulting anion with a borate compound.
